(19) United States
(12) Patent Application Publication
(10) Pub. No.: US 2025/0306046 A1
(43) Pub. Date: Oct. 2, 2025

(54) METHOD FOR ESTABLISHING METROLOGICAL TRACEABILITY FOR AT LEAST ONE IN VITRO DIAGNOSTIC MEDICAL DEVICE

(71) Applicant: Roche Diagnostics Operations, Inc., Indianapolis, IN (US)

(72) Inventors: Andrea Geistanger, Muenchen (DE); Anton Hilger, Muenchen (DE); Daniel Intelmann, Schaftlach (DE)

(21) Appl. No.: 18/862,932

(22) PCT Filed: May 4, 2023
(86) PCT No.: PCT/EP2023/061804

§ 371 Date: Nov. 4, 2024

(30) Foreign Application Priority Data
May 4, 2022 (EP) 22171611.1

(51) Int. Cl. G01N 35/00; G16H 40/40

(52) U.S. Cl. CPC G01N 35/00693; G16H 40/40

(57) ABSTRACT

Methods for establishing metrological traceability for at least one in vitro diagnostic medical device (110) are proposed. The methods comprise a sequence of calibration steps and adjustment steps. An outcome of each step depends on the outcome of the previous step. The methods comprises providing a leading calibration curve. The leading calibration curve fₚ describes a relationship of at least one concentration c of at least one analyte in at least one sample with a signal s of the sample measured with the in vitro diagnostic medical device (110). The leading calibration curve fₚ is a parametrized function fₚ(c, \hat{p}₁, . . . , \hat{p}_{P}) with parameters \hat{p}₁, . . . , \hat{p}_{P} being a set of parameters of the leading calibration curve and P≥1. In each adjustment step a signal adjustment function or a concentration adjustment function is determined and at least one target concentration value is assigned.

### TECHNICAL FIELD

The invention relates to methods for establishing metrological traceability for at least one in vitro diagnostic medical device (IVD MD), a processing device, a kit comprising an in vitro diagnostic medical device and a set of IVD MD calibrators and their target concentration values, a computer program and a computer program product.

### BACKGROUND ART

Generally, the goal of calibration procedures of mass spectrometry devices is to transfer trueness from a higher order reference, e.g. a reference measurement procedure or reference material, to an analytical application, also referred to as an assay, by means of target value assignment of calibrators. Usually, such an approach establishes a traceability chain for each measurement of a patient sample to the higher order reference and makes analytical applications and their results comparable around the world. The general process is well described, e.g. in ISO 17511:2020.

In order to transfer trueness from one method to another method, dedicated samples, e.g. patient samples, or calibrators, are often used. As a rule, these samples are value assigned in the higher order method and are used for calibration of the lower order method. The obtained calibration function usually depends on various factors such as: individual instrument, hardware parts, e.g. in case of hardware multiplexing, reagent lot, individual reagent, e.g. within a lot, new set of calibration samples, the calibration event itself and time effects. As a consequence, every individual calibration function may generally vary in their functional parameters. Methods described in the prior art usually include multiple individual and independent calibration steps. This means, that the variance of each step may generally contribute completely to the overall variance of the whole method. A higher variance usually makes the calibration approach less robust and increases uncertainty.

WO 2021/239692 A1 describes a computer implemented method for calibrating a customer mass spectrometry instrument for quantifier-qualifier-ratio check. The method comprises the following steps: a) at least one manufacturer-site standardization, wherein a set of samples of a subject and a set of calibrator samples are measured in multiple replicates on a plurality of mass spectrometry instruments, wherein each measurement comprises multiple reaction monitoring with quantifier and qualifier transition for analyte and internal standard, wherein at least three adjustment factors are determined from the measurements of the set of samples of a subject and the set of calibrator samples, wherein a first adjustment factor; depends on a difference between analyte and internal standard, wherein a second adjustment factor; depends on a difference between samples of a subject and calibrator samples for analyte quantifier-qualifier-ratio, wherein a third adjustment factor; depends on a difference between samples of a subject and calibrator samples for the internal standard quantifier-qualifier-ratio; b) at least one transfer step, wherein the adjustment factors are electronically transferred to a customer mass spectrometry instrument; c) at least one customer-site calibration, wherein the customer-site calibration comprises at least one calibration measurement, wherein a set of calibrator samples is measured on the customer mass spectrometry instrument and quantifier-qualifier-ratios are determined therefrom, wherein target values for quantifier-qualifier-ratios for analyte and for internal standard are set by applying the adjustment factors on the determined quantifier-qualifier-ratios.

EP 3 472 624 B1 describes a method for providing a calibration curve for an optical D-dimer assay.

### Problem to be Solved

It is therefore an objective of the present invention to provide methods for establishing metrological traceability for at least one in vitro diagnostic medical device, a processing device, a kit comprising an in vitro diagnostic medical device and a set of IVD MD calibrators and their target concentration values, a computer program and a computer program product, which avoid the above-described disadvantages of known methods, devices, computer programs and computer program products. In particular, the method and devices shall minimize or reduce the overall variance of the calibration process. Specifically, the calibration process shall be optimized, in particular by increasing a robustness of the calibration process and/or by reducing an uncertainty of the calibration process.

### SUMMARY

This problem is addressed by methods for establishing metrological traceability for at least one in vitro diagnostic medical device, a processing device, a kit comprising an in vitro diagnostic medical device and a set of IVD MD calibrators and their target concentration values, a computer program and a computer program product with the features of the independent claims. Advantageous embodiments, which may be realized in an isolated fashion or in any arbitrary combinations, are listed in the dependent claims as well as throughout the specification.

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements.

Further, it shall be noted that the terms “at least one”, “one or more” or similar expressions indicating that a feature or element may be present once or more than once typically will be used only once when introducing the respective feature or element. In the following, in most cases, when referring to the respective feature or element, the expressions “at least one” or “one or more” will not be repeated, non-withstanding the fact that the respective feature or element may be present once or more than once.

Further, as used in the following, the terms “preferably”, “more preferably”, “particularly”, “more particularly”, “specifically”, “more specifically” or similar terms are used in conjunction with optional features, without restricting alternative possibilities. Thus, features introduced by these terms are optional features and are not intended to restrict the scope of the claims in any way. The invention may, as the skilled person will recognize, be performed by using alternative features. Similarly, features introduced by “in an embodiment of the invention”or similar expressions are intended to be optional features, without any restriction regarding alternative embodiments of the invention, without any restrictions regarding the scope of the invention and without any restriction regarding the possibility of combining the features introduced in such way with other optional or non-optional features of the invention.

In a first aspect of the present invention, a method for establishing metrological traceability for at least one in vitro diagnostic medical device is disclosed.

The method may be computer-implemented. The term “computer implemented” as used herein is a broad term and is to be given its ordinary and customary meaning to a person of ordinary skill in the art and is not to be limited to a special or customized meaning. The term specifically may refer, without limitation, to a method involving at least one computer and/or at least one computer network. The computer and/or computer network may comprise at least one processor which is configured for performing at least one of the method steps of the method according to the present invention. Preferably each of the method steps is performed by the computer and/or computer network. The method may be performed completely automatically, such as without user interaction. The term “automatically” as used herein is a broad term and is to be given its ordinary and customary meaning to a person of ordinary skill in the art and is not to be limited to a special or customized meaning. The term specifically may refer, without limitation, to a process which is performed completely by means of at least one computer and/or computer network and/or machine, in particular without manual action and/or interaction with a user.

The term “metrological traceability” as used herein is a broad term and is to be given its ordinary and customary meaning to a person of ordinary skill in the art and is not to be limited to a special or customized meaning. The term specifically may refer, without limitation, to a property of a measurement result whereby the result can be related to a reference through a documented unbroken chain of calibration and adjustment steps.

The term “in vitro diagnostic medical device” (IVD MD) as used herein is a broad term and is to be given its ordinary and customary meaning to a person of ordinary skill in the art and is not to be limited to a special or customized meaning. The term specifically may refer, without limitation, to a medical device, whether used alone or in combination, which is configured for in vitro examination of at least one sample derived from the human body, and/or configured for providing information for diagnostic, monitoring or compatibility purposes. The IVD MD may comprise one or more of at least one reagent, at least one calibrator, at least one control material, at least one specimen receptacle, software, related instruments or apparatus or other articles.

The in vitro diagnostic medical device may be a mass spectrometry device. The term “mass spectrometry” as used herein is a broad term and is to be given its ordinary and customary meaning to a person of ordinary skill in the art and is not to be limited to a special or customized meaning. The term specifically may refer, without limitation, to an analytical technique for determining a mass-to-charge ratio of ions. The mass spectrometry may be performed using at least one mass spectrometry device. As used herein, the term “mass spectrometry device”, also denoted “mass analyzer”, is a broad term and is to be given its ordinary and customary meaning to a person of ordinary skill in the art and is not to be limited to a special or customized meaning. The term specifically may refer, without limitation, to an analyzer configured for detecting at least one analyte based on the mass-to-charge ratio.

The mass analyzer may be or may comprise at least one quadrupole mass analyzer. As used herein, the term “quadrupole mass analyzer” is a broad term and is to be given its ordinary and customary meaning to a person of ordinary skill in the art and is not to be limited to a special or customized meaning. The term specifically may refer, without limitation, to a mass analyzer comprising at least one quadrupole as mass filter. The quadrupole mass analyzer may comprise a plurality of quadrupoles. For example, the quadrupole mass analyzer may be a triple quadrupole mass spectrometer. As used herein, the term “mass filter” is a broad term and is to be given its ordinary and customary meaning to a person of ordinary skill in the art and is not to be limited to a special or customized meaning. The term specifically may refer, without limitation, to a device configured for selecting ions injected to the mass filter according to their mass-to-charge ratio m/z. The mass filter may comprise two pairs of electrodes. The electrodes may be rod-shaped, e.g. cylindrical. In ideal case, the electrodes may be hyperbolic. The electrodes may be designed identical. The electrodes may be arranged in parallel extending along a common axis, e.g. a z axis. The quadrupole mass analyzer may comprise at least one power supply circuitry configured for applying at least one direct current (DC) voltage and at least one alternating current (AC) voltage between the two pairs of electrodes of the mass filter. The power supply circuitry may be configured for holding each opposing electrode pair at identical potential. The power supply circuitry may be configured for changing sign of charge of the electrode pairs periodically such that stable trajectories are only possible for ions within a certain mass-to-charge ratio m/z. Trajectories of ions within the mass filter can be described by the Mathieu differential equations. For measuring ions of different m/z values DC and AC voltage may be changed in time such that ions with different m/z values can be transmitted to a detector of the mass spectrometry device.

The mass spectrometry device may further comprise at least one ionization source. As used herein, the term “ionization source”, also denoted as “ion source”, is a broad term and is to be given its ordinary and customary meaning to a person of ordinary skill in the art and is not to be limited to a special or customized meaning. The term specifically may refer, without limitation, to a device configured for generating ions, e.g. from neutral gas molecules. The ionization source may be or may comprise at least one source selected from the group consisting of: at least one gas phase ionization source such as at least one electron impact (EI) source or at least one chemical ionization (CI) source; at least one desorption ionization source such as at least one plasma desorption (PDMS) source, at least one fast atom bombardment (FAB) source, at least one secondary ion mass spectrometry (SIMS) source, at least one laser desorption (LDMS) source, and at least one matrix assisted laser desorption (MALDI) source; at least one spray ionization source such as at least one thermospray (TSP) source, at least one atmospheric pressure chemical ionization (APCI) source, at least one electrospray (ESI), and at least one atmospheric pressure ionization (API) source.

The mass spectrometry device may comprise at least one detector. As used herein, the term “detector”, is a broad term and is to be given its ordinary and customary meaning to a person of ordinary skill in the art and is not to be limited to a special or customized meaning. The term specifically may refer, without limitation, to an apparatus configured for detecting incoming ions. The detector may be configured for detecting charged particles. The detector may be or may comprise at least one electron multiplier. The mass spectrometry device, e.g. the detector and/or at least one processing unit of the mass spectrometry device, may be configured to determine at least one mass spectrum of the detected ions. As used herein, the term “mass spectrum” is a broad term and is to be given its ordinary and customary meaning to a person of ordinary skill in the art and is not to be limited to a special or customized meaning. The term specifically may refer, without limitation, to a two dimensional representation of signal intensity vs the charge-to-mass ratio m/z, wherein the signal intensity corresponds to abundance of the respective ion. The mass spectrum may be a pixelated image. For determining resulting intensities of pixels of the mass spectrum, signals detected with the detector within a certain m/z range may be integrated. The analyte in the sample may be identified by the processing unit. The processing unit may be configured for correlating known masses to the identified masses or through a characteristic fragmentation pattern.

The mass spectrometry device may be or may comprise a liquid chromatography mass spectrometry device. The mass spectrometry device may be connected to and/or may comprise at least one liquid chromatograph. The liquid chromatograph may be used as sample preparation for the mass spectrometry device. Other embodiments of sample preparation may be possible, such as at least one gas chromatograph. As used herein, the term “liquid chromatography mass spectrometry device” is a broad term and is to be given its ordinary and customary meaning to a person of ordinary skill in the art and is not to be limited to a special or customized meaning. The term specifically may refer, without limitation, to a combination of liquid chromatography with mass spectrometry. The mass spectrometry device may comprise at least one liquid chromatograph. The liquid chromatography mass spectrometry device may be or may comprise at least one high performance liquid chromatography (HPLC) device or at least one micro liquid chromatography (μLC) device. The liquid chromatography mass spectrometry device may comprise a liquid chromatography (LC) device and a mass spectrometry (MS) device, in the present case the mass filter, wherein the LC device and the mass filter are coupled via at least one interface. The interface coupling the LC device and the MS device may comprise the ionization source configured for generating of molecular ions and for transferring of the molecular ions into the gas phase. The interface may further comprise at least one ion mobility module arranged between the ionization source and the mass filter. For example, the ion mobility module may be a high-field asymmetric waveform ion mobility spectrometry (FAIMS) module.

As used herein, the term “liquid chromatography (LC) device” is a broad term and is to be given its ordinary and customary meaning to a person of ordinary skill in the art and is not to be limited to a special or customized meaning. The term specifically may refer, without limitation, to an analytical module configured to separate one or more analytes of interest of a sample from other components of the sample for detection of the one or more analytes with the mass spectrometry device. The LC device may comprise at least one LC column. For example, the LC device may be a single-column LC device or a multi-column LC device having a plurality of LC columns. The LC column may have a stationary phase through which a mobile phase is pumped in order to separate and/or elute and/or transfer the analytes of interest. The liquid chromatography mass spectrometry device may further comprise a sample preparation station for the automated pre-treatment and preparation of samples each comprising at least one analyte of interest.

The term “analyte”, as used herein, relates to any chemical compound or group of compounds which shall be determined in a sample. The analyte detected by the mass spectrometry device may be part of a sample, e.g. a solid, liquid, or gaseous sample, which is examined, e.g. measured, with the mass spectrometry device. As a result of the measurement process, the mass spectrometry device may detect a presence and/or an abundance and/or a concentration of one or more analytes, e.g. a plurality of analytes, in the sample. The analyte may be a sample component as such. Additionally or alternatively, the analyte may be a fragment of a component present in the sample. As an example, one or more of the sample components may be fragmented during the measurement process, e.g. during an ionization procedure, such that a single sample component may yield a plurality of different fragments, e.g. charged fragments, which may at least partially be detected as analytes by the mass spectrometry device.

For example, the analyte may be a macromolecule, i.e. a compound with a molecular mass of more than 1000 u (i.e. more than 1 kDa). For example, the analyte may be a biological macromolecule, e.g. a polypeptide, a polynucleotide, a polysaccharide, or a fragment of any of the aforesaid. For example, the analyte may be a small molecule chemical compound, i.e. a compound with a molecular mass of at most 1000 u (1 kDa). For example, the analyte may be a chemical compound metabolized by a body of a subject, e.g. of a human subject, or may be a compound administered to a subject in order to induce a change in the subject's metabolism. Thus, for example, the analyte may be a drug of abuse or a metabolite thereof, e.g. amphetamine; cocaine; methadone; ethyl glucuronide; ethyl sulfate; an opiate, for example buprenorphine, 6-monoacatylmorphine, codeine, dihydrocodeine, morphine, morphine-3-glucuronide, and/or tramadol; and/or an opioid, for example acetylfentanyl, carfentanil, fentanyl, hydrocodone, norfentanyl, oxycodone, and/or oxymorphone.

For example, the analyte may be a therapeutic drug, e.g. valproic acid; clonazepam; methotrexate; voriconazole; mycophenolic acid (total); mycophenolic acid-glucuronide; acetaminophen; salicylic acid; theophylline; digoxin; an immuno suppressant drug, for example cyclosporine, everolimus, sirolimus, and/or tacrolimus; an analgesic, for example meperidine, normeperidine, tramadol, and/or O-desmethyl-tramadol; an antibiotic, for example gentamycin, tobramycin, amikacin, vancomycin, piperacilline (tazobactam), meropenem, and/or linezolid; an antieplileptic, for example phenytoin, valporic acid, free phenytoin, free valproic acid, levetiracetam, carbamazepine, carbamazepine-10,11-epoxide, phenobarbital, primidone, gabapentin, zonisamid, lamotrigine, and/or topiramate. For example, the analyte may be a hormone, such as cortisol, estradiol, progesterone, testosterone, 17-hydroxyprogesterone, aldosterone, dehydroepiandrosteron (DHEA), dehydroepiandrosterone sulfate (DHEA-S), dihydrotestosterone, and/or cortisone; for example, the sample may be a serum or plasma sample and the analyte may be cortisol, DHEA-S, estradiol, progesterone, testosterone, 17-hydroxyprogesterone, aldosterone, DHEA, dihydrotestosterone, and/or cortisone; for example, the sample may be a saliva sample and the analyte may be cortisol, estradiol, progesterone, testosterone, 17-hydroxyprogesterone, androstendione, and/or cortisone; for example, the sample may be a urine sample and the analyte may be cortisol, aldosterone, and/or cortisone. For example, the analyte may be a vitamin, for example vitamin D, e.g. ergocalciferol (Vitamin D2) and/or cholecalciferol (Vitamin D3) or a derivative thereof, e.g. 25-hydroxy-vitamine-D2, 25-hydroxy-vitamine-D3, 24,25-dihydroxy-vitamine-D2, 24,25-dihydroxy-vitamine-D3, 1,25-dihydroxy-vitamine-D2, and/or 1,25-dihydroxy-vitamine-D3. For example, the analyte may be a metabolite of a subject.

The in vitro diagnostic medical device may comprise at least one hardware part. The in vitro diagnostic medical device may comprise a plurality of hardware parts. The term “hardware part” is a broad term and is to be given its ordinary and customary meaning to a person of ordinary skill in the art and is not to be limited to a special or customized meaning. The term specifically may refer, without limitation, to a physical and/or tangible part of the in vitro diagnostic medical device. The hardware parts may be configured to interact with another, e.g. in order to fulfill at least one common function of the in vitro diagnostic medical device. The hardware parts may be handled independently or may be coupled, connectable or integratable with each other. For example, the hardware part may be or may comprise an instrument or a component that forms part of the in vitro diagnostic medical device such as of the mass spectrometry device, e.g. of one or more of: a sample preparation unit of the mass spectrometry device, an ionization unit of the mass spectrometry device, a mass analyzer unit of the mass spectrometry device and a detection unit of the mass spectrometry device. The hardware part may have a specific configuration or setting that may be variable or adjustable, e.g. in an application-specific manner. Additionally or alternatively, the configuration or the setting may vary due to manufacturing tolerances. For example, due to the potential variability of the hardware part, a calibration of the hardware part may be required.

As used herein, the term “sample”, also referred to as “test sample”, may relate to any type of composition of matter; thus, the term may refer, without limitation, to any arbitrary sample such as a biological sample and/or an internal standard sample. For example, the sample may be a liquid sample, e.g. an aqueous sample. For example, the test sample may be selected from the group consisting of: a physiological fluid, including whole blood, serum, plasma, saliva, ocular lens fluid, lacrimal fluid, cerebrospinal fluid, sweat, urine, milk, ascites, mucus, synovial fluid, peritoneal fluid, and amniotic fluid; lavage fluid; tissue, cells, or the like. The sample may, however, also be a natural or industrial liquid, e.g. surface or ground water, sewage, industrial wastewater, processing fluid, soil eluates, and the like. For example, the sample may comprise or may be suspected to comprise at least one chemical compound of interest, i.e. a chemical which shall be determined, which is referred to as “analyte”. The sample may comprise one or more further chemical compounds, which are not to be determined and which are commonly referred to as matrix, as specified herein above. The sample may be used directly as obtained from the respective source or may be subjected to one or more pretreatment and/or a sample preparation step(s). Thus, the sample may be pretreated by physical and/or chemical methods, for example by centrifugation, filtration, mixing, homogenization, chromatography, precipitation, dilution, concentration, contacting with a binding and/or detection reagent, and/or any other method deemed appropriate by the skilled person.

In, i.e. before, during, and/or after, the sample preparation step, one or more internal standard(s) may be added to the sample. The sample may be spiked with the internal standard. For example, an internal standard may be added to the sample at a predefined concentration. The internal standard may be selected such that it is easily identifiable under normal operating conditions of the detector chosen, e.g. a mass spectrometry device, a photometric cell, e.g. in an UV-Vis spectroscopic device, an evaporative light scattering refractometer, a conductometer, or any device deemed appropriate by the skilled person. The concentration of the internal standard may be pre-determined and significantly higher than the concentration of the analyte. For example, analytes of interest may be vitamin D, drugs of abuse, therapeutic drugs, hormones, and metabolites in general. The internal standard sample may be a sample comprising at least one internal standard substance with a known concentration. For further details with respect to the sample, reference is made e.g. to EP 3 425 369 A1, the full disclosure is included herewith by reference. Other analytes of interest are possible.

The method comprises a sequence of calibration and adjustment steps. An outcome of each step depends on the outcome of the previous step. This may allow for establishing metrological traceability for the in vitro diagnostic medical device.

The method comprises providing a leading calibration curve. The leading calibration curve fₚ describes a relationship of at least one concentration c of at least one analyte in at least one sample with a signal s of the sample measured with the in vitro diagnostic medical device. The leading calibration curve fₚ is a parametrized function fₚ(c, \hat{p}₁, . . . , \hat{p}_{P}) with parameters \hat{p}₁, . . . , \hat{p}_{P} being a set of parameters of the leading calibration curve and P≥1.

In each adjustment step a signal adjustment function gᵣ with r being a set of parameters of the signal adjustment function, describing a relationship between measured and theoretical signal values, is determined by determining the relationship between measured signal values of first calibrator samples and theoretical signal values of the first calibrator samples derived from the leading calibration curve. The theoretical signal values are determined by applying the leading calibration curve using pre-assigned target concentration values cᵢ of the first calibrator samples.

Each adjustment step comprises assigning at least one target concentration value from measured signal values of at least one second calibrator sample. The assigning comprises determining at least one theoretical signal value of the second calibrator sample by applying the signal adjustment function determined in the previous adjustment step or the inverse of the signal adjustment function determined in the previous adjustment step to the measured signal values of the second calibrator sample and applying the inverse leading calibration curve f_{P}⁻¹ to the theoretical signal value of the second calibrator sample.

The method steps and/or substeps of the method steps, e.g. the above-described actions comprised in each of the method steps, may, for example, be performed in the given order. However, a different order may also be possible. The method may further comprise additional method steps, which are not listed. Further, one or more or even all of the method steps and/or the substeps, may be performed only once or repeatedly.

As outlined above, the method according to the present invention uses a sequence of calibration and adjustment steps such that stablishing metrological traceability for the in vitro diagnostic medical device is possible. In particular, the method according to the present invention solves the problem of transferring trueness from a higher order reference, e.g. a reference measurement procedure or reference material, to an analytical application. A known process is described in ISO 17511:2020, wherein in each step a specific measurement procedure and a specific material is used, whose target values were assigned in a preceding step. However, in the known process of ISO 17511:2020, multiple individual and independent calibration steps are used. This means, that the variance of each step contributes completely to the overall variance of the whole process. The present invention proposes a different approach for establishing a traceability chain, i.e. using a sequence of calibration and adjustment steps, wherein an outcome of each step depends on the outcome of the previous step. The traceability chain can be reached as follows. A leading calibration curve is used (the leading calibration curve fₚ describes a relationship of a concentration c of an analyte in a sample with a signal s of the sample measured with the IVD device) and said leading calibration curve is maintained unchanged during the whole chain of processes. Instead of having different calibrations (and thus, different calibration curves) for each step of the chain, adjustment steps are proposed, in which a signal adjustment function is determined, which describes a relationship between measured and theoretical signal values. Each adjustment step may comprise two steps, e.g. sub steps:
determining the signal adjustment function by using measured signal values of first calibrator samples and using theoretical signal values of the first calibrator samples derived from the leading calibration curve; the theoretical signal values are determined by applying the leading calibration curve using pre-assigned target concentration values cᵢ of the first calibrator samples;
assigning target concentration values from measured signal values of a second calibrator sample are assigned (which can be used in a subsequent step). This can be done by using the determined signal adjustment function (or the invers), which is applied to the measured signal values of the second calibrator sample, thereby determining theoretical signal values of the second calibrator sample. Then, the inverse leading calibration curve can be applied to the determined theoretical signal value of the second calibrator sample thereby determining the target concentration values.

These two adjustment (sub) steps, i.e. determining the signal adjustment function and target value assignment for calibrators which are used in a subsequent adjustment step as “preassigned target concentration values”, can be performed for each process of the chain, thereby reaching traceability.

The term “calibration step” is a broad term and is to be given its ordinary and customary meaning to a person of ordinary skill in the art and is not to be limited to a special or customized meaning. The term specifically may refer, without limitation, to an arbitrary process of determining a relationship between measured signals generated by the in vitro diagnostic medical device, e.g. upon sample examination, and corresponding analyte concentration values of the sample. The method may comprise a plurality of calibration steps. Each of the calibration steps may comprise one or several steps that may e.g. be performed repeatedly or that may be complemented by further steps, such as to enhance or refine the calibration. For example, a calibration step may comprise providing a leading calibration curve, e.g. a relationship between measured signals generated by the in vitro diagnostic medical device and corresponding analyte concentration values of the sample.

The term “adjustment step” is a broad term and is to be given its ordinary and customary meaning to a person of ordinary skill in the art and is not to be limited to a special or customized meaning. The term specifically may refer, without limitation, to a process of determining a signal adjustment function. The method may comprise a plurality of adjustment steps. Each of the adjustment steps may comprise one or several steps that may e.g. be performed repeatedly or that may be complemented by further steps, such as to enhance or refine the adjustment. For example, the adjustment step may comprise determining of the signal adjustment function using the first calibrator samples, as well as the assigning of target concentration values to the second calibrator samples.

The sequence of calibration and adjustment steps may comprise at least one calibration step, e.g. in which a leading calibration curve is provided, and at least one adjustment step, e.g. in which a signal adjustment function is determined and target concentration values are assigned to calibrator samples. The adjustment step in this sequence may succeed the calibration step, since it uses the theoretical signal values of the first calibrator samples, which are derived from the leading calibration curve. The sequence of steps may comprise additional calibration steps and/or adjustment steps. For example, subsequently, a second, a third and a fourth adjustment step may be performed.

The sequence of calibration and adjustment steps may comprise a plurality of calibration and/or adjustment steps. The method may comprise a whole standardization procedure. An outcome of each step depends on the outcome of the previous step. In the following the expression calibration and adjustment step (in singular or plural form) is used for denoting a step of the standardization procedure. The method may comprise a hierarchy of calibration and adjustment steps. The sequence of calibration and adjustment steps may comprise performing method steps from a reference to the final measuring system, where the outcome of each step depends on the outcome of the previous step. The method may comprise establishing metrological traceability by ensuring traceability to higher order reference system components as required by ISO 17511:2020. The metrological traceability may refer to the hierarchy of calibration and adjustment steps and a sequence of value assignments, which may allow an unbroken linkage between a measurement result for the sample up to the highest available reference system component in the hierarchy.

Each of the calibration and adjustment steps may comprise using at least one measurement procedure. The term “measurement procedure” is a broad term and is to be given its ordinary and customary meaning to a person of ordinary skill in the art and is not to be limited to a special or customized meaning. The term specifically may refer, without limitation, to a detailed description of a measurement according to one or more measurement principles and to a given measurement method based on a measurement model and including any calculation to obtain a measurement result. The measurement procedure may be performed on at least one material, wherein the material is specified according to the respective measurement procedure. The measurement procedure used in the respective calibration step and the respective adjustment step may be performed as described in ISO 17511:2020.

For example, the sequence of calibration and adjustment steps comprises a first calibration and adjustment step using a fit for purpose measurement procedure for purity assessment, e.g. quantitative NMR, mass balance or the like. Different measurement procedures may serve as primary reference measurement procedure, e.g. measurement procedures based on gas chromatography-mass spectrometry (GC/MS) or liquid chromatography coupled with tandem mass spectrometry (LC-MS/MS). Other methods are feasible.

The sequence of calibration and adjustment steps may further comprise a second calibration and adjustment step using a primary reference measurement procedure for calibrator preparation, e.g. gravimetric preparation, on at least one certified primary reference material. The primary reference measurement procedure may be or may comprise a reference measurement procedure used to obtain a measurement result without relation to a measurement standard for a quantity of the same kind. The term “certified reference material (CRM)” is a broad term and is to be given its ordinary and customary meaning to a person of ordinary skill in the art and is not to be limited to a special or customized meaning. The term specifically may refer, without limitation, to a reference material accompanied by documentation issued by an authoritative body and providing one or more specified property values with associated uncertainties and traceabilities, using valid procedures. The primary reference measurement procedure for calibrator preparation and the CRM may fulfill the requirements described in ISO 17511:2020 and ISO 15194. The target concentration value for the CRM may be assigned by the first calibration and adjustment step.

The sequence of calibration and adjustment steps may further comprise a third calibration and adjustment step using a primary reference measurement procedure for a measurand on at least one primary calibrator. The term “measurand” is a broad term and is to be given its ordinary and customary meaning to a person of ordinary skill in the art and is not to be limited to a special or customized meaning. The term specifically may refer, without limitation, to a quantity intended to be measured. The term “calibrator” is a broad term and is to be given its ordinary and customary meaning to a person of ordinary skill in the art and is not to be limited to a special or customized meaning. The term specifically may refer, without limitation, to material used as measurement standard. The primary calibrator may be a measurement standard established using a primary reference measurement procedure, or created as an artefact, chosen by convention. The primary calibrator may be prepared as solution of the CRM in a suitable solvent. The primary reference measurement procedure for a measurand and the primary calibrator may fulfill the requirements described in ISO 17511:2020. The target concentration value for the primary calibrator may be assigned by the second calibration and adjustment step.

The sequence of calibration and adjustment steps may further comprise a forth calibration and adjustment step using a manufacturer selected measurement procedure on at least one secondary calibrator. The secondary calibrator may be a measurement standard established through calibration with respect to a primary measurement standard for a quantity of the same kind. The manufacturer selected measurement procedure and the secondary calibrator may fulfill the requirements described in ISO 17511:2020. The secondary calibrator may be at least one of: human samples, pools of human samples, samples with matrix and samples comparable to human samples. The target concentration value for the secondary calibrator may be assigned by the third calibration and adjustment step. The term “manufacturer” is a broad term and is to be given its ordinary and customary meaning to a person of ordinary skill in the art and is not to be limited to a special or customized meaning. The term specifically may refer, without limitation, to an entity with responsibility for design, manufacture, fabrication, assembly, packaging or labelling of an IVD MD, for assembling a measuring system, or adapting an IVD MD before it is placed on the market and/or put into service, regardless of whether these operations are carried out by that entity or on their behalf by a third party. The manufacturer's selected measurement procedure may comprise one or more of homogenous or heterogeneous immunoassays or liquid chromatography coupled with tandem mass spectrometry (LC-MS/MS). Other measurement procedures are feasible. The manufacturer's selected measurement procedure may be at least partially automated. The manufacturer's selected measurement procedure may for example be comparable to or attuned to a customer's measurement procedure.

As outlined above, the method comprises providing a leading calibration curve. The term “leading calibration curve” as used herein is a broad term and is to be given its ordinary and customary meaning to a person of ordinary skill in the art and is not to be limited to a special or customized meaning. The term specifically may refer, without limitation, to an arbitrary mathematical function describing a relationship of at least one concentration c of at least one analyte in at least one sample with a signal s of the sample measured with the mass spectrometry device. The leading calibration curve may comprise at least one mathematical operation, e.g. a multiplication with at least one factor or any other type of mathematical operation. The leading calibration curve is a parametrized function. The term “parametrized function” is a broad term and is to be given its ordinary and customary meaning to a person of ordinary skill in the art and is not to be limited to a special or customized meaning. The term specifically may refer, without limitation, to an arbitrary mathematical function having at least one parameter, e.g. a plurality of parameters such as a set of at least two parameters. The leading calibration curve fₚ is a parametrized function fₚ(c, \hat{p}₁, . . . , \hat{p}_{P}) with parameters \hat{p}₁, . . . , \hat{p}_{P} being a set of parameters of the leading calibration curve and P≥1. The term “parameter” is a broad term and is to be given its ordinary and customary meaning to a person of ordinary skill in the art and is not to be limited to a special or customized meaning. The term specifically may refer, without limitation, to an arbitrary quantity, which influences an output or a behavior of a mathematical function and which is viewed as being held constant. The parameter may be configured for determining a behavior of the mathematical function. A variable of a mathematical function, in contrast, may be viewed as changing. The term “set of parameters” may generally refer to a plurality of parameter of a single mathematical function. The leading calibration curve may be formed, for example determined, by a functional form of the leading calibration curve together with fitted parameters values \hat{p}₁, . . . , \hat{p}_{P}. The functional form may e.g. be one or more of a Rodbart model function, a Padé model function, a quadratic model function, or any other non-linear or linear function. For example, Padé model function may be given by

$$γ = \frac{{p}_{1} + {p}_{2} \cdot x}{1 + {p}_{3} \cdot x} ,$$

where y is signal value, x is concentration or target value and p₁, p₂ and p₃ are the parameters of the function. For example, Rodbard model function is given by

$$y = \frac{{p}_{1} - {p}_{4}}{1 + {( \frac{x}{{p}_{2}} )}^{P 3}} + {p}_{4} ,$$

where y is signal value, x is concentration or target value and p₁, p₂, p₃ and p₄ are the parameters of the function. Other functional forms are also feasible.

The term “providing a leading calibration curve”, is a broad term and is to be given its ordinary and customary meaning to a person of ordinary skill in the art and is not to be limited to a special or customized meaning. The term specifically may refer, without limitation, to one or more of determining the leading calibration curve by at least one of setting and choosing the leading calibration curve. For example, the process of setting the leading calibration curve may comprise establishing the leading calibration curve, e.g. by determining at least one of its form and/or at least one of the parameter values \hat{p}₁, . . . , \hat{p}_{P}, such as by choosing at least one model function and/or by fitting at least one of the parameter values. The setting of the leading calibration curve may, for example, comprise generating at least one specific parameter value in a modelling and/or fitting procedure.

The leading calibration curve, in particular the inverse leading calibration curve, may assign a concentration c to a sample examined by the in vitro diagnostic medical device on the basis of at least one theoretical signal value derived from a measured signal value. Additionally or alternatively, the leading calibration curve, e.g. the inverse leading calibration curve, may assign a concentration c to a sample examined by the in vitro diagnostic medical device on the basis of at least one measured signal value, which was measured by the in vitro diagnostic medical device. Additionally or alternatively, the leading calibration curve may contribute to assigning a concentration c to a sample, by assigning a theoretical concentration value to a sample on the basis of the measured signal value of the sample, wherein the concentration c is assigned to the sample on the basis of the theoretical concentration value in a further step.

The leading calibration curve may be an assay and/or application-specific function. For example, multiple conditions may be used for its determination, e.g. one or more of different instruments, different reagent lots and different measurement conditions that may be used in the assay or application. For example, the leading calibration curve may be determined by using multiple conditions such as one or more of multiple instruments, and/or hardware parts and/or reagent lots and the like. The providing of the leading calibration curve may be part of at least one of the calibration and adjustment steps. The providing of the leading calibration curve may be performed at a high order step of the hierarchy, e.g. one of the first steps in the hierarchy.

For example, the leading calibration curve may be determined by using at least one primary calibrator. At least one target concentration value of the primary calibrator may be established based on a primary reference measurement procedure for a calibrator preparation. For example, the leading calibration curve is provided by using at least one secondary calibrator. At least one target concentration value of the secondary calibrator may be established based on the primary reference measurement procedure for a measurand. Other examples for providing of the leading calibration function in other steps of the hierarchy are also feasible. Additionally or alternatively, the leading calibration curve may be provided by retrieving a leading calibration curve such as from at least one database, e.g. a cloud. The providing of the leading calibration curve may be performed once or repeatedly.

The term “target concentration value”, is a broad term and is to be given its ordinary and customary meaning to a person of ordinary skill in the art and is not to be limited to a special or customized meaning. The term specifically may refer, without limitation, to an assigned concentration value, e.g. a concentration value that is pre-assigned in a higher order calibration and adjustment step.

The term “signal” as used herein is a broad term and is to be given its ordinary and customary meaning to a person of ordinary skill in the art and is not to be limited to a special or customized meaning. The term specifically may refer, without limitation, to an arbitrary electronic signal generated by the in vitro diagnostic medical device and/or at least one hardware part upon detection of the at least one analyte. The signal may be a measurement signal of the in vitro diagnostic medical device. The signal may be the measured signal on the in vitro diagnostic medical device and/or hardware part j, with repeat 1, with j≥1 and l≥0. The generation of the signal may be part of the measurement process. For example, in case of a mass spectrometry device, the signal or a set of signals may be interpreted to determine, e.g. identify, the detected analyte and/or its concentration. The signals may be plotted in a mass spectrum as a function of the mass-to-charge ratio of the analyte.

The term “concentration” as used herein is a broad term and is to be given its ordinary and customary meaning to a person of ordinary skill in the art and is not to be limited to a special or customized meaning. The term may refer, without limitation, to an abundance of a component or constituent in a given volume, such as the sample volume, set in relation to said volume, e.g. divided by the volume. The placeholder “c” may stand for the concentration. The concentration may be described by different kinds of quantities such as by a mass concentration, by a molar concentration, or by a volume concentration. It shall be noted that the term concentration may quantify components or constituents present in the volume of a liquid sample as well as in the volume of a solid or gaseous sample. The concentration of the analyte may be specified and/or quantified by a concentration value.

A relationship between the measured signal values of calibrator samples and the target concentration values of said calibrator samples may be described only insufficiently, such as not precisely or accurately enough, by the leading calibration curve. As an example, the concentration values as determined using the leading calibration curve on the basis of the measured signal values of said calibrator samples may differ from the target concentration values of said calibrator samples as by more than a pre-determined threshold value. For example, this may result from the fact that the leading calibration curve was determined on the basis of samples other than said calibrator samples. In the standardization process, the leading calibration curve may nevertheless be kept unchanged and, instead, a further function, the signal adjustment function may be determined and/or adjusted. While the standardization process comprises several calibration and adjustment steps, the leading calibration curve may be established initially, such as in a high order step of the hierarchy, and may be kept unchanged over the two or more subsequent adjustment steps. As an example, possible subsequent adjustments and/or refinements may be taken into account by the signal adjustment function described in more detail below.

In each adjustment step a signal adjustment function gᵣ with r being a set of parameters of the signal adjustment function is determined. The term “signal adjustment function” as used herein is a broad term and is to be given its ordinary and customary meaning to a person of ordinary skill in the art and is not to be limited to a special or customized meaning. The term specifically may refer, without limitation, to an arbitrary mathematical function describing a relationship between the measured signal values of samples and theoretical signal values of the samples. The signal adjustment function may be a linear or non-linear function. The signal adjustment function may be a parametrized function. The signal adjustment function may be written as g(sᵢ^{theo}, r₁, . . . , r_{R}) with r₁, . . . , r_{R} being a set of parameters of the signal adjustment function, R≥1 and i being the first calibrator samples i=1, . . . . I, I≥1.

The functional form of the signal adjustment function may e.g. be one or more of a Rodbart model function, a Padé model function, a quadratic model function, or any other non-linear or linear function. For example, Padé model function may be given by

$$y = \frac{{p}_{1} + {p}_{2} \cdot x}{1 + {p}_{3} \cdot x} ,$$

where y is signal value, x is concentration or target value and p₁, p₂ and p₃ are the parameters of the function. For example, Rodbard model function is given by

$$y = \frac{{p}_{1} - {p}_{4}}{1 + {( \frac{x}{{p}_{2}} )}^{P 3}} + {p}_{4} ,$$

where y is signal value, x is concentration or target value and p₁, p₂, p₃ and p₄ are the parameters of the function. Other functional forms are also feasible.

As described above, the signal adjustment function describes a relationship between measured and theoretical signal values. The indicated relationship may be described by a function mapping the measured signal values to the theoretical signal values just as well as by a function mapping the theoretical signal values to the measured signal values. Both these functions may be regarded as signal adjustment functions. Depending on the choice when determining the signal adjustment function, either the signal adjustment function or its inverse may be applied to determine the theoretical signal value from the measured signal values.

The signal adjustment function or the inverse of the signal adjustment function may be applied to the measured signal values of the second calibrator sample in order to determine at least one theoretical signal value of the second calibrator sample.

The term “first calibrator sample” as used herein is a broad term and is to be given its ordinary and customary meaning to a person of ordinary skill in the art and is not to be limited to a special or customized meaning. The term specifically may refer, without limitation, to a calibrator with pre-assigned target concentration values. The first calibrator samples may comprise at least one set of calibrator samples comprising a plurality of calibrator samples. The target concentration values of the first calibrator samples may be assigned by a higher order step of the hierarchy. For example, the target concentration values may be pre-assigned in the previous adjustment step. Different first calibrator samples may be used in different subsequent adjustment steps.

The first calibrator samples may be measured with the in vitro diagnostic medical device, e.g. the mass spectrometry device, to determine measured signal values of the first calibrator samples. The signal adjustment function may connect theoretical signals of the leading calibration curve sᵢ^{theo}=f(cᵢ, \hat{p}1, . . . , \hat{p}_{P}), with i≥1 and cᵢ being the pre-assigned target concentration values of the first calibrator samples, with measured signal values sᵢⱼₗ^{meas} of the first calibrator samples, wherein j denotes an instrument and/or hardware part of the in vitro diagnostic medical device j=1, . . . , J and J≥1, and 1 the repeat l=1, . . . , L, and L≥1. The determining of the signal adjustment function may comprise measuring the signal values sᵢⱼₗ^{meas} of the first calibrator samples, calculating the theoretical signals sᵢ^{theo} and fitting the signal adjustment function thereby determining the fitted parameters \hat{r}₁, . . . , \hat{r}_{R}. The leading calibration curve and the signal adjustment function may complement each other in connecting measured signal values of samples with target concentration values of the at least one analyte in the sample.

The term “theoretical signal values” as used herein is a broad term and is to be given its ordinary and customary meaning to a person of ordinary skill in the art and is not to be limited to a special or customized meaning. The term specifically may refer, without limitation, to calculated signal values from a given centration value. The theoretical signal values of the first calibrator samples may be determined by applying the leading calibration curve to the pre-assigned target concentration values cᵢ of the first calibrator samples.

Having measured the signal values of the first calibrator samples and having determined the theoretical signal values of the first calibrator samples the signal adjustment function can be determined.

Each adjustment step comprises assigning at least one target concentration value from measured signal values of at least one second calibrator sample. The method may comprise assigning target concentration values of the second calibrator samples for use in a subsequent calibration an adjustment step. The term “second calibrator sample” as used herein is a broad term and is to be given its ordinary and customary meaning to a person of ordinary skill in the art and is not to be limited to a special or customized meaning. The term specifically may refer, without limitation, to a calibrator different from the first calibrator sample. The method may comprise measuring signal values of at least one second calibrator sample. The second calibrator may have an unknown target concentration value. The signal values for the second calibrator sample may be measured with each individual hardware part. Theoretical signal values may be assigned by applying the inverse signal adjustment function to the measured signal values. The theoretical signal values may be transformed into target concentration values, by applying the inverse leading calibration curve f_{P}⁻¹ to the theoretical signal value of the second calibrator sample. For example, the assigning of the target concentration value may comprise measuring signal values sₖⱼₗ^{meas} of the second calibrator sample k, with k=1, . . . , K and K≥1, wherein j denotes an instrument and/or hardware part j=1, . . . , J and J≥1, and l the repeat l=1, . . . , L, and L≥1, using the in vitro diagnostic medical device and transforming the measured signal values sₖⱼₗ^{meas} into target concentration values cₖⱼₗ by applying the following inverse functions consecutively:

$${s}_{kjl}^{theo} = {g}_{1}^{- 1} ( {s}_{kjl}^{meas} , {( {\hat{r}}_{1} , … , {\hat{r}}_{R} )}_{j} ) ; and$$

$${c}_{kjl} = {f}^{- 1} ( {s}_{kjl}^{theo} , {\hat{p}}_{1} , … , {\hat{p}}_{P} ) .$$

The target concentration value of the second calibrator sample may be assigned as at least one averaged value from a plurality of target concentration values, wherein the average value is one or more of mean, median or weighted mean value of the plurality of target concentration values. The plurality of target concentration values may, for example, result from repetitions of at least one of the adjustment steps. Weights for the weighted mean value may e.g. be given by an inverse of a variability of the repeats performed on a specific instrument and/or hardware parts.

The signal adjustment function may connect measured signals values of the first calibrator samples with the theoretical signals of the first calibrator samples derived from the pre-assigned target concentrations and the leading calibration function. The assignment of the target concentration values of the second calibrator sample may be determined by applying the signal adjustment function.

The measured signal values of the second calibrator samples may be used for assigning target concentration values to the second calibrator samples. Such an assignment may be performed using the signal adjustment function (or its inverse) and the leading calibration function as described in detail above. In other words, the method according to the present invention proposes adjusting the measured signal values to theoretical signal values, which may be translated into the target concentration values using the leading calibration curve.

The standardization process may comprise a plurality of adjustment steps such that more than one signal adjustment function, e.g. two or even more signal adjustment functions, may be determined during the standardization step and/or the previously determined signal adjustment function may be adjusted during the standardization step. During the standardization process the signal adjustment function may be refined or adjusted to specific conditions. As an example, a first signal adjustment function may be complemented and/or replaced by a second signal adjustment function and so on.

The method comprises a plurality of successively performed calibration and adjustment steps. For example, the method may comprise one or more of the following steps: setting of the leading calibration curve; adjustment of the leading calibration curve; target value assignment of leading calibrators; adjustment of the leading calibration curve by leading calibrators; target value assignment of product calibrators. The steps may be performed at the manufacturer's side.

As outlined above, the step of providing the leading calibration curve may be embodied as comprising the setting of the leading calibration curve. For example, this step may be performed as one of the initial steps of the standardization process.

The step of setting of the leading calibration curve may comprise the determining of the functional form of the leading calibration curve. The step of setting of the leading calibration curve may comprise the determining of the parameter values of the parameters \hat{p}₁, . . . , \hat{p}_{P} of the leading calibration curve fₚ(c, \hat{p}₁, . . . , \hat{p}_{P}).

For example, the method may comprise at least one step of setting of the leading calibration curve. The step may comprise measuring signal values sᵢⱼₗ^{meas} of a set of primary calibrators with the in vitro diagnostic medical device, with i denoting the primary calibrators i=1, . . . , l and l≥1, j denoting the instrument and/or hardware part j=1, . . . , J and J≥1, and l denoting the repeat l=1, . . . , L and L≥1, and determining parameter values of the parameters \hat{p}₁, . . . , \hat{p}_{P} of the leading calibration curve fₚ(c, \hat{p}₁, . . . , \hat{p}_{P}) using pre-assigned target concentration values cᵢ of the set of primary calibrators and the measured signal values sᵢⱼᵢ^{meas}. The set of primary calibrator may comprise at least one primary calibrator, such as at least three primary calibrators, e.g. 20 to 30 primary calibrators. The set of primary calibrators may, however also comprise a different number of primary calibrators, e.g. more than 30 primary calibrators. The target concentration values cᵢ of the primary calibrators may be pre-assigned in a higher order calibration and/or adjustment step, e.g. comprising the primary reference measurement procedure for a calibrator preparation. The primary reference measurement procedure may be, for example, based on gas chromatography-mass spectrometry (GC/MS) or liquid chromatography coupled with tandem mass spectrometry (LC-MS/MS). Other techniques are possible.

For example, the method may comprise at least one step of setting of the leading calibration curve. The step may comprise measuring signal values sᵢⱼₗ^{meas} of a set of secondary calibrators with the in vitro diagnostic medical device, with i denoting the secondary calibrators i=1, . . . , I and I≥1, j denoting the instrument and/or hardware part j=1, . . . , J and J≥1, and l denoting the repeat l=1, . . . , L and L≥1, and determining parameter values of the parameters \hat{p}₁, . . . , \hat{p}_{P} of the leading calibration curve fₚ(c, \hat{p}₁, . . . , \hat{p}_{P}) using pre-assigned target concentration values cᵢ of the set of secondary calibrators and the measured signal values sᵢⱼₗ^{meas}. The set of secondary calibrator may comprise at least one secondary calibrator, such as at least three secondary calibrators, e.g. 20 to 30 secondary calibrators. The set of secondary calibrators may, however also comprise a different number of secondary calibrators, e.g. more than 30 secondary calibrators. The target concentration values cᵢ of the secondary calibrators may be pre-assigned in a higher order calibration and/or adjustment step, e.g. comprising a primary reference measurement procedure for a measurand.

Subsequently to the providing of the leading calibration curve, e.g. by setting the leading calibration curve using primary or secondary calibrators or retrieving the leading calibration curve, the method may further comprise at least one step of adjustment of the leading calibration curve comprising determining a first signal adjustment function. For example, as outlined above, the providing of the leading calibration curve may comprise using primary calibrators. The method may comprise at least one step of adjustment of the leading calibration curve comprising determining a first signal adjustment function g₁(sᵢ^{theo}, r₁, . . . , r_{R}) using a set of secondary calibrators i=1, . . . . I, I≥1, wherein the determining of the first signal adjustment function comprises
measuring signal values sᵢⱼₗ^{meas} of the secondary calibrators using a manufacturer's selected measurement procedure,
calculating the theoretical signals sᵢ^{theo} by

$${s}_{i}^{theo} = f ( {c}_{i} , {\hat{p}}_{1} , … , {\hat{p}}_{P} ) ,$$

with cᵢ being the pre-determined target concentration values of the secondary calibrators, and
fitting the signal adjustment function g₁ thereby determining the fitted parameters \hat{r}₁, . . . , \hat{r}_{R}.

The step of adjustment of the leading calibration curve may, for example, comprise determining the first signal adjustment function, while keeping the leading calibration curve unchanged.

The pre-assigned target concentration values of the secondary calibrators may be determined in a higher order calibration and/or adjustment step, e.g. comprising a primary reference measurement procedure for a measurand.

In the subsequent calibration and adjustment step the two functions, i.e. the leading calibration function and the first signal adjustment function, may be used in conjunction to assign at least one target concentration value to a sample such as the leading calibrators. The target concentration value may be assigned to the sample on the basis of the leading calibration curve and at least one signal adjustment function, e.g. by consecutively applying the inverse of the signal adjustment function to the measured signal value to determine the theoretical signal value and the inverse of the leading calibration curve to the theoretical signal value.

The method may comprise at least one step of target value assignment of leading calibrators. The “leading calibrator” as used herein is a broad term and is to be given its ordinary and customary meaning to a person of ordinary skill in the art and is not to be limited to a special or customized meaning. The term specifically may refer, without limitation, to a measurement standard that is used to calibrate or verify measuring instruments or measuring systems. The leading calibrators may be samples, which do not need to be so close to the human sample matrix as the primary and secondary calibrators. The leading calibrators may correspond to the samples denoted as manufacturer's working calibrator in the standard ISO 17511:2020.

As an example, the step of target value assignment of leading calibrators may initially be carried out in direct subsequence to the step of setting of the leading calibration curve. In this case, no signal adjustment function may have been determined yet and thus, no signal adjustment function may be available. The target concentration values may be assigned to the leading calibrators using the leading calibration curve alone, e.g. by applying the inverse of the leading calibration curve to the measured signal values of the leading calibrator. Alternatively, the step of target value assignment of leading calibrators may be carried out in a repetition of steps, such that the step of adjustment of the leading calibration curve, which in the hierarchy of calibration and adjustment steps succeeds the step of target value assignment of leading calibrators, has already been carried out. For the repetition of the step of target value assignment of leading calibrators, the signal adjustment function may be available. In this case, the step of target values assignment of leading calibrators may be carried out using both the leading calibration curve and the signal adjustment function.

For example, the step of target value assignment of leading calibrators may comprise assigning at least one target concentration value cₖⱼₗ^{lead} of at least one leading calibrator. The assigning of the target concentration value cₖⱼₗ^{lead} of the leading calibrator may comprise measuring signal values sₖⱼₗ^{lead,meas} of the leading calibrator k with k=1, . . . , K, and K≥1 using the in vitro diagnostic medical device, with j denoting the instrument and/or hardware part j=1, . . . , J and J≥1, and/denoting the repeat l=1, . . . , L and L≥1. The assigning of the target concentration value cₖⱼₗ^{lead} of the leading calibrator may further comprise transforming the measured signal values sₖⱼₗ^{lead,meas}, into target concentration values cₖⱼₗ^{lead} by applying the following inverse functions consecutively:

$${s}_{kjl}^{lead , theo} = {g}_{1}^{- 1} ( {s}_{kjl}^{lead , meas} , {( {\hat{r}}_{1} , … , {\hat{r}}_{R} )}_{j} ) ; and$$

$${c}_{kjl}^{lead} = {f}^{- 1} ( {s}_{kjl}^{lead , theo} , {\hat{p}}_{1} , … , {\hat{p}}_{P} ) .$$

The target concentration value of the leading calibrator may e.g. be assigned as at least one averaged value from a plurality of target concentration values, wherein the average value is one or more of mean, median or weighted mean value of the plurality of target concentration values.

The method may further comprise checking whether the assignment of the target concentration values of the leading calibrators was successful. The method may further comprise measuring signal values of independent control samples using the in vitro diagnostic medical device, e.g. the mass spectrometry device. In order to check, whether the assignment of the target concentration values of the leading calibrators was successful, the method may comprise transforming the measured signal values of the independent control samples into concentration values by applying the signal adjustment function g₁ and the leading calibration curve and comparing the concentration values to pre-defined target concentration values of the independent control samples. The independent control samples may have target values concₘ^{cont}, m=1, . . . , M₁, M₁≥1 established by a higher order method. If either the absolute or relative deviation of the read concentration to the target values of the independent control samples stays within predefined limits, the target values of the new calibrators may be accepted. The method may further comprise comparing the concentration values to pre-defined target concentration values of the independent control samples, wherein the target concentration values cₖⱼₜ^{lead} are accepted if a deviation between the concentration values of the independent control samples and the pre-defined target concentration values of the independent control samples is within a predefined tolerance range.

The method may comprise at least one step of adjustment of the leading calibration curve using the at least one leading calibrator k. The step of adjustment of the leading calibration curve may comprise determining a second signal adjustment function g₂(sₖ^{theo}, q₁, . . . , q_{Q}) with q₁, . . . , q_{Q} being a set of parameters of the second signal adjustment function and Q≥1, by
measuring signal values sₖⱼₗ^{lead,meas} of the at least one leading calibrator k using a manufacturer's standing measurement procedure on the in vitro diagnostic medical device, with j denoting the instrument and/or hardware part j=1, . . . , J and J≥1, and I denoting the repeat l=1, . . . , L and L≥1,
calculating the theoretical signals sₖ^{theo} by

$${s}_{k}^{theo} = f ( {c}_{kjl}^{lead} , {\hat{p}}_{1} , … , {\hat{p}}_{P} ) , and$$

fitting the signal adjustment function g₂ thereby determining the fitted parameters \hat{q}₁, . . . , \hat{q}_{Q}.

The manufacturer's standing measurement procedure may comprise e.g. at least one of homogenous immunoassays, heterogeneous immunoassays and liquid chromatography coupled with tandem mass spectrometry (LC-MS/MS). Other measurement procedures are feasible. The manufacturer's standing measurement procedure may be at least partially automated. The manufacturer's standing measurement procedure may, for example, be comparable to or attuned to a customer's measurement procedure.

The step of adjustment of the leading calibration curve by leading calibrators may, for example, comprise determining the signal adjustment function g₂ using the measured signal values of the leading calibrators, while keeping the leading calibration curve itself unchanged. In the following the two functions, i.e. the leading calibration function and the second signal adjustment function, may be used in conjunction to assign the at least one target concentration value of at least one further second calibrator sample, e.g. of product calibrators as described in further detail below.

The method may further comprise at least one step of target value assignment of product calibrators. The product calibrators may correspond to samples denoted as end user in vitro diagnostic medical device calibrators in the standard ISO 17511:2020. The step of target value assignment of product calibrators may comprise assigning at least one target concentration value cₖⱼₗ^{pr} of at least one product calibrator k. The assigning of the target concentration value cₖⱼₗ^{pr} of the product calibrator comprises measuring signal values sₖⱼₗ^{pr,meas} of the product calibrator k with k=1, . . . , K, and K≥1 using the in vitro diagnostic medical device, with j denoting the instrument and/or hardware part j=1, . . . J and J≥1, and l denoting the repeat l=1, . . . , L and L≥1, wherein the determining of the target concentration value cₖⱼₗ^{pr} of the product calibrator further comprises transforming the measured signal values sₖⱼₗ^{pr,meas}, into target concentration values cₖⱼₗ^{pr} by applying the following inverse functions consecutively:

$${s}_{kjl}^{pr , theo} = {g}_{2}^{- 1} ( {s}_{kjl}^{pr , meas} , {( {\hat{q}}_{1} , … , {\hat{q}}_{Q} )}_{j} ) , and$$

$${c}_{kjl}^{pr} = {f}^{- 1} ( {s}_{kjl}^{pr , theo} , {\hat{p}}_{1} , … , {\hat{p}}_{P} ) .$$

The target concentration value of the product calibrator may e.g. be assigned as at least one averaged value from a plurality of target concentration values, wherein the average value is one or more of mean, median or weighted mean value of the plurality of target concentration values.

The method may further comprise checking whether the assignment of the target concentration values of the product calibrators was successful. The method may further comprise measuring signal values of independent control samples using the in vitro diagnostic medical device, e.g. the mass spectrometry device, and transforming the measured signal values of the independent control samples into concentration values by applying the signal adjustment function g₂ and the leading calibration curve. The method may further comprise comparing the concentration values to pre-assigned target concentration values of the independent control samples. The target concentration values cₖⱼₗ^{pr} may be accepted if a deviation between the concentration values of the independent control samples and the pre-assigned target concentration values of the independent control samples is within a predefined tolerance range.

The method may further comprise performing at least one customer-side calibration step. For example, the product calibrators with accepted target concentration values cₖⱼₗ^{pr} may be used for the customer-side calibration step.

In a further aspect of the present invention, a method for establishing metrological traceability for at least one in vitro diagnostic medical device is disclosed. The method comprises a sequence of calibration steps and adjustment steps. An outcome of each step depends on the outcome of the previous step. The method comprises providing a leading calibration curve, wherein the leading calibration curve fₚ describes a relationship of at least one concentration c of at least one analyte in at least one sample with a signal s of the sample measured with the in vitro diagnostic medical device, wherein the leading calibration curve fₚ is a parametrized function fₚ(c, \hat{p}₁, . . . , \hat{p}_{P}) with parameters \hat{p}₁, . . . , \hat{p}_{P} being a set of parameters of the leading calibration curve and P≥1. In each adjustment step a concentration adjustment function hₛ with s being a set of parameters of the concentration adjustment function, describing a relationship between a theoretical concentration of measured signal values of first calibrator samples and an pre-assigned target concentration value of the first calibrator samples, is determined, wherein the theoretical concentration of the first calibrator samples is determined by applying the inverse of the leading calibration curve f_{P}⁻¹ using the measured signal values of the first calibrator samples. The assignment of the target concentration value comprises applying the inverse of the leading calibration curve f_{P}⁻¹ using measured signal values of second calibrator samples, thereby obtaining theoretical concentration values, and applying the concentration adjustment function hₛ⁻ or the inverse of the concentration adjustment function hₛ⁻¹ on said theoretical concentration values, thereby obtaining the target concentration values of the second calibrator samples.

With respect to definitions and embodiments of the method described in a further aspect, reference is made to definitions and embodiments of the method described in a first aspect of the present invention.

The term “concentration adjustment function” as used herein is a broad term and is to be given its ordinary and customary meaning to a person of ordinary skill in the art and is not to be limited to a special or customized meaning. The term specifically may refer, without limitation, to an arbitrary mathematical function describing a relationship between target concentration values of samples and theoretical concentration values of the samples. The concentration adjustment function may be a linear or non-linear function. The concentration adjustment function may be a parametrized function. The functional form of the concentration adjustment function may e.g. be one or more of a Rodbart model function, a Padé model function, a quadratic model function, or any other non-linear or linear function. For example, Padé model function may be given by

$$γ = \frac{{p}_{1} + {p}_{2} \cdot x}{1 + {p}_{3} \cdot x} ,$$

where y is signal value, x is concentration or target value and p₁, p₂ and p₃ are the parameters of the function. For example, Rodbard model function is given by

$$y = \frac{{p}_{1} - {p}_{4}}{1 + {( \frac{x}{{p}_{2}} )}^{P 3}} + {p}_{4} ,$$

where y is signal value, x is concentration or target value and p₁, p₂, p₃ and p₄ are the parameters of the function. Other functional forms are also feasible.

The concentration adjustment function may connects pre-assigned target concentration of the first calibrator samples with the theoretical concentration of the measured signal of the first calibrator samples and the assignment of the target concentration values of second calibrator samples may be determined by applying the concentration adjustment function.

In a further aspect of the present invention, a processing device is disclosed. The processing device configured for retrieving and/or storing at least one pre-determined leading calibration curve fₚ. The processing device is further configured for storing a set of parameters \hat{p}₁, . . . , \hat{p}_{P} of the leading calibration curve fₚ, wherein P is a positive integer. The leading calibration curve fₚ describes a relationship of at least one concentration c of at least one analyte in at least one sample with a signal s of the sample measured with the in vitro diagnostic medical device. The leading calibration curve fₚ is a parametrized function fₚ(c, \hat{p}₁, . . . , \hat{p}_{P}). The processing device is further configured for performing one or both of the method for establishing metrological traceability for at least one in vitro diagnostic medical device according to the present invention.

The leading calibration curve is pre-determined on the manufacture's side by using one or both of the methods according to the present invention e.g. according to any one of the embodiments described above and/or according to any one of the embodiments described in further detail below. Thus, for possible details and options of the method as well as for terms and definitions, reference may be made to the description of the method for establishing metrological traceability for at least one in vitro diagnostic medical device as given above or as further given below.

The term “processing device” as used herein is a broad term and is to be given its ordinary and customary meaning to a person of ordinary skill in the art and is not to be limited to a special or customized meaning. The term specifically may refer, without limitation, to a device or a combination of devices configured for controlling at least one function of at least one other device, such as of at least one other component of the in vitro diagnostics medical device, e.g. the mass spectrometry device. The processing device may e.g. comprise at least one processor and/or at least one data storage device. Thus, as an example, the at least one processing device may comprise at least one data processing device having a software code stored thereon comprising a number of computer commands. The processing device may be an element of an in vitro diagnostics medical device or a further device, e.g. a remote device.

The processing device may further be configured for conducting at least one customer-side calibration step for the IVD medical device on at least one set of product calibrators thereby adjusting the signal adjustment function. The customer-side calibration step may comprise
measuring signal values sₖⱼₗ^{pr,meas} of the at least one product calibrator k using a customer-side measurement procedure on the IVD medical device, with j denoting the instrument and/or hardware part j=1, . . . , J and J≥1, and/denoting the repeat l=1, . . . , L and L≥1,
calculating the theoretical signals sₜ^{theo} by

$${s}_{k}^{theo} = f ( {c}_{kjl}^{pr} , {\hat{p}}_{1} , … , {\hat{p}}_{P} ) ,$$

with cₖⱼₗ^{pr} being the pre-assigned target concentration values of the set of product calibrators, wherein the target concentration values were pre-assigned on the manufacturer's side by using one or both methods according to any one of the embodiments described above and/or according to any one of the embodiments described in further detail below,
fitting the signal adjustment function thereby determining the fitted parameters of the signal adjustment function.

The processing device may further be configured for conducting at least one customer-side calibration step for the IVD medical device on at least one set of product calibrators thereby adjusting the concentration adjustment function hₛ. The customer-side calibration step may comprise measuring signal values sₖⱼₗ^{pr,meas} of the at least one product calibrator k using a customer-side measurement procedure on the IVD medical device, with j denoting the instrument and/or hardware part j=1, . . . , J and J≥1, and/denoting the repeat l=1, . . . , L and L≥1,
calculating the theoretical concentration values signals cₖ^{theo} by

$${c}_{k}^{theo} = {f}^{- 1} ( {s}_{kjl}^{pr , meas} ) ,$$

fitting the concentration adjustment function using the calculated theoretical concentration values and pre-assigned target concentration values cₖⱼₗ^{pr}, thereby determining the fitted parameters of the concentration adjustment function, wherein the pre-assigned target concentration values cₖⱼₗ^{pr} were pre-assigned on the manufacturer's side by using one or both methods according to any one of the embodiments described above and/or according to any one of the embodiments described in further detail below.

In a further aspect of the present invention, a kit comprising the in vitro diagnostic medical device, e.g. the mass spectrometry device, and a set of product calibrators is disclosed. The target concentration values of the product calibrators are determined by using one or both of the methods according to the present invention. For possible details regarding the kit as well as for terms and definitions, reference may be made to the description of the methods as given above or as further given below.

For example, the kit may be provided with the following information:
The functional form of the leading calibration curve f and the parameters \hat{p}₁, . . . , \hat{p}_{P}, P≥1. These parameters may be specific for an assay, but independent of the other components of the measurement system, as the instrument, reagent lot or calibrator lot. Hence, they are once transferred on the in vitro diagnostic medical device and do not need adjustments, when changes on other system components occur.
The functional form of the signal-adjustment function on the in vitro diagnostic medical device. This function may have another functional form as the signal-adjustment functions within standardization.
A set of K₂ product calibrators, with K2≥1, with assigned target values from step “target value assignment of product calibrators”.

This information may allow calibrating the in vitro diagnostic medical device at the costumer's side. With this information the individual instrument can be re-calibrated and sample reading can take place, e.g. as described in WO 2021/122739.

In a further aspect of the present invention, a computer program is disclosed, wherein the computer program is adapted to perform the methods according to any one of the embodiments described above and/or according to any one of the embodiments described in further detail below while the program is being executed on a computer. Specifically, the computer program may include computer-executable instructions for performing the method when the instructions are executed on a computer or computer network. Specifically, the computer program may be stored on a computer-readable data carrier and/or on a computer-readable storage medium.

As used herein, the terms “computer-readable data carrier” and “computer-readable storage medium” specifically may refer to non-transitory data storage means, such as a hardware storage medium having stored thereon computer-executable instructions. The computer-readable data carrier or storage medium specifically may be or may comprise a storage medium such as a random-access memory (RAM) and/or a read-only memory (ROM).

Thus, specifically, one, more than one or even all of method steps as indicated above, specifically the calibration and/or adjustment steps, may be performed by using a computer or a computer network, preferably by using a computer program.

In a further aspect of the present invention, a computer program product having program code means is disclosed, wherein the program code means can be stored or are stored on a storage medium, for performing the methods according to any one of the embodiments described above and/or according to any one of the embodiments described in further detail below, when the program code means are executed on a computer or on a computer network. Specifically, the program code means may be stored on a computer-readable data carrier and/or on a computer-readable storage medium.

As used herein, a computer program product refers to the program as a tradable product. The product may generally exist in an arbitrary format, such as in a paper format, or on a computer-readable data carrier and/or on a computer-readable storage medium. Specifically, the computer program product may be distributed over a data network.

Further disclosed and proposed herein is a data carrier having a data structure stored thereon, which, after loading into a computer or computer network, such as into a working memory or main memory of the computer or computer network, may execute the method according to one or more of the embodiments disclosed herein.

Further disclosed and proposed herein is a non-transient computer-readable medium including instructions that, when executed by one or more processors, cause the one or more processors to perform the method according to one or more of the embodiments disclosed herein.

Finally, disclosed and proposed herein is a modulated data signal, which contains instructions readable by a computer system or computer network, for performing the method according to one or more of the embodiments disclosed herein.

Referring to the computer-implemented aspects of the invention, one or more of the method steps or even all of the method steps of the method according to one or more of the embodiments disclosed herein may be performed by using a computer or computer network. Thus, generally, any of the method steps including provision and/or manipulation of data may be performed by using a computer or computer network. Generally, these method steps may include any of the method steps, typically except for method steps requiring manual work, such as providing the samples and/or certain aspects of performing the actual measurements. Specifically, the steps of providing the leading calibration curve, determining the signal adjustment function and/or assigning the at least one target concentration value may be performed by using the computer or computer network.

Summarizing and without excluding further possible embodiments, the following embodiments may be envisaged:

Embodiment 1: A method for establishing metrological traceability for at least one in vitro diagnostic medical device, wherein the method comprises a sequence of calibration and adjustment steps, wherein an outcome of each step depends on the outcome of the previous step,
wherein the method comprises providing a leading calibration curve, wherein the leading calibration curve fₚ describes a relationship of at least one concentration c of at least one analyte in at least one sample with a signal s of the sample measured with the in vitro diagnostic medical device, wherein the leading calibration curve fₚ is a parametrized function fₚ(c, \hat{p}₁, . . . , \hat{p}_{P}) with parameters \hat{p}₁, . . . , \hat{p}_{P} being a set of parameters of the leading calibration curve and P≥1,
wherein in each adjustment step a signal adjustment function gᵣ with r being a set of parameters of the signal adjustment function, describing a relationship between measured and theoretical signal values, is determined by determining the relationship between measured signal values of first calibrator samples and theoretical signal values of the first calibrator samples derived from the leading calibration curve, wherein the theoretical signal values are determined by applying the leading calibration curve using pre-assigned target concentration values cᵢ of the first calibrator samples,
wherein each adjustment step comprises assigning at least one target concentration value from measured signal values of at least one second calibrator sample, wherein the assigning comprises determining at least one theoretical signal value of the second calibrator sample by applying the signal adjustment function determined in the previous adjustment step or the inverse of the signal adjustment function determined in the previous adjustment step to the measured signal values of the second calibrator sample and applying the inverse leading calibration curve f_{P}⁻¹ to the theoretical signal value of the second calibrator sample.

Embodiment 2: The method according to the preceding embodiment, wherein the sequence of calibration and adjustment steps comprises a first calibration and adjustment step using a fit for purpose measurement procedure for purity assessment, wherein the sequence of calibration and adjustment steps further comprises a second calibration and adjustment step using a primary reference measurement procedure for calibrator preparation on at least one certified primary reference material, wherein the sequence of calibration and adjustment steps further comprises a third calibration and adjustment step using a primary reference measurement procedure for a measurand on at least one primary calibrator, and a forth calibration and adjustment step using a manufacturer selected measurement procedure on at least one secondary calibrator.

Embodiment 3: The method according to the preceding embodiment, wherein the leading calibration curve is determined by using at least one primary calibrator, wherein at least one target concentration value of the primary calibrator is established based on the primary reference measurement procedure for a calibrator preparation.

Embodiment 4: The method according to any one of the two preceding embodiments, wherein the leading calibration curve is determined by using at least one secondary calibrator, wherein at least one target concentration value of the secondary calibrator is established based on the primary reference measurement procedure for a measurand.

Embodiment 5: The method according to any one of the preceding embodiments, wherein the leading calibration curve is determined by using multiple conditions such as one or more of multiple instruments and/or hardware parts and/or reagent lots.

Embodiment 6: The method according to any one of the preceding embodiments, wherein the leading calibration curve is unchanged over two or more adjustment steps.

Embodiment 7: The method according to any one of the preceding embodiments, wherein a functional form of the leading calibration curve together with fitted parameter values \hat{p}₁, . . . , \hat{p}_{P} form the leading calibration curve, wherein the functional form of the leading calibration curve is one or more of a Rodbart model function, a Padé model function, a quadratic model function, or any other non-linear or linear function.

Embodiment 8: The method according to any one of the preceding embodiments, wherein the signal adjustment function is a function g(sᵢ^{theo}, r₁, . . . , r_{R}) with r₁, . . . , r_{R} being a set of parameters of the signal adjustment function, R≥1 and i being the first calibrator samples i=1, . . . . I, I≥1, wherein the signal adjustment function connects theoretical signals of the leading calibration curve sᵢ^{theo}=f(cᵢ, \hat{p}₁, . . . , \hat{p}_{P}), with i≥1 and cᵢ being the preassigned target concentration values of the first calibrator samples, with measured signal values sᵢⱼₗ^{meas} of the first calibrator samples, wherein j denotes an instrument and/or hardware part j=1, . . . , J and J≥1, and 1 the repeat l=1, . . . , L, and L≥1, wherein the determining of the signal adjustment function comprises measuring the signal values sᵢⱼₗ^{meas} of the first calibrator samples, calculating the theoretical signals sᵢ^{theo} and fitting the signal adjustment function thereby determining the fitted parameters \hat{r}₁, . . . , \hat{r}_{R}.

Embodiment 9: The method according to any one of the preceding embodiments, wherein the assignment of the target concentration value comprises measuring signal values sₖⱼₗ^{meas} of the second calibrator sample k, with k=1, . . . , K and K≥1 using the in vitro diagnostic medical device and transforming the measured signal values sₖⱼₗ^{meas} into target concentration values cₖⱼₗ by applying the following inverse functions consecutively:

$${s}_{kjl}^{theo} = {g}_{1}^{- 1} ( {s}_{kjl}^{meas} , {( {\hat{r}}_{1} , … , {\hat{r}}_{R} )}_{j} ) ; and$$

$${c}_{kjl} = {f}^{- 1} ( {s}_{kjl}^{theo} , {\hat{p}}_{1} , … , {\hat{p}}_{p} ) .$$

Embodiment 10: The method according to any one of the preceding embodiments, wherein the signal adjustment function is a function g(sᵢ^{theo}, r₁, . . . , r_{R}) with r₁, . . . , r_{R} being a set of parameters of the signal adjustment function, R≥1 and i being the first calibrator samples i=1, . . . . I, I≥1, wherein the signal adjustment function connects measured signal values of the first calibrator samples with the theoretical signals of the first calibrator samples derived from the pre-assigned target concentrations and the leading calibration function, wherein the assignment of the target concentration values of the second calibrator sample is determined by applying the signal adjustment function.

Embodiment 11: The method according to any one of the preceding embodiments, wherein a functional form of the signal adjustment function together with fitted parameter values \hat{r}₁, . . . , \hat{r}_{R} form the signal adjustment function, wherein the functional form of the signal adjustment function is one or more of a Rodbart model function, a Padé model function, a quadratic model function, or any other non-linear or linear function.

Embodiment 12: The method according to any one of the preceding embodiments, wherein the pre-assigned target concentration values are pre-assigned in a previous adjustment step.

Embodiment 13: The method according to any one of the preceding embodiments, wherein the target concentration value of the second calibrator sample is assigned as at least one averaged value from a plurality of target concentration values, wherein the average value is one or more of mean, median or weighted mean value of the plurality of target concentration values.

Embodiment 14: The method according to any one of the preceding embodiments, wherein the in vitro diagnostic medical device is a mass spectrometry device.

Embodiment 15: The method according to any one of the preceding embodiments, wherein the method comprises one or more of the following steps: setting of the leading calibration curve; adjustment of the leading calibration curve; target value assignment of leading calibrators; adjustment of the leading calibration curve by leading calibrators; target value assignment of product calibrators, wherein the steps are performed at the manufacturer's side.

Embodiment 16: The method according to the preceding embodiment, wherein the method comprises at least one step of setting of the leading calibration curve comprising measuring signal values sᵢⱼₗ^{meas} of a set of reference samples with the in vitro diagnostic medical device, such as the mass spectrometry device, using a primary reference measurement procedure, with i denoting the reference samples i=1, . . . , I and I≥1, j denoting the instrument and/or hardware part j=1, . . . , J and/≥1, and l denoting the repeat l=1, . . . , L and L≥1, and determining parameter values of the parameters \hat{p}₁, . . . , \hat{p}_{P} of the leading calibration curve fₚ(c, \hat{p}₁, . . . , \hat{p}_{P}) using pre-assigned target concentration values cᵢ of the set of reference samples and the measured signal values sᵢⱼₗ^{meas}.

Embodiment 17: The method according to the preceding embodiment, wherein the method comprises at least one step of adjustment of the leading calibration curve comprising determining a first signal adjustment function g₁(sᵢ^{theo}, r₁, . . . , r_{R}) using a set of secondary calibrators i=1, . . . . I, I≥1,
wherein the determining of the first signal adjustment function comprises
   measuring signal values sᵢⱼₗ^{meas} of the secondary calibrators using a manufacturer's selected measurement procedure on the in vitro diagnostic medical device, such as the mass spectrometry device,
   calculating the theoretical signals sᵢ^{theo} by

$${s}_{i}^{theo} = f ( {c}_{i} , {\hat{p}}_{1} , … , {\hat{p}}_{P} ) ,$$

with cᵢ being the pre-assigned target concentration values of the secondary calibrators, and fitting the signal adjustment function g₁ thereby determining the fitted parameters \hat{r}₁, . . . , \hat{r}_{R}.

Embodiment 18: The method according to the preceding embodiment, wherein the method comprises at least one step of target value assignment of leading calibrators comprising
assigning at least one target concentration value cₖⱼₗ^{lead} of at least one leading calibrator, wherein the assigning of the target concentration value cₖⱼₗ^{lead} of the leading calibrator comprises measuring signal values sₖⱼₗ^{lead,meas} of the leading calibrator k with k=1, . . . , K, and K≥1 using the in vitro diagnostic medical device, such as the mass spectrometry device, with j denoting the instrument and/or hardware part j=1, . . . , J and J≥1, and l denoting the repeat l=1, . . . , L and L≥1, wherein the assigning of the target concentration value, cₖⱼₗ^{lead} of the leading calibrator further comprises transforming the measured signal values sₖⱼₗ^{lead,meas}, into target concentration values cₖⱼₗ^{lead} by applying the following inverse functions consecutively:

$${s}_{kjl}^{lead , theo} = {g}_{1}^{- 1} ( {s}_{kjl}^{lead , meas} , {( {\hat{r}}_{1} , … , {\hat{r}}_{R} )}_{j} ) ; and$$

$${c}_{kjl}^{lead} = {f}^{- 1} ( {s}_{kjl}^{lead , theo} , {\hat{p}}_{1} , … , {\hat{p}}_{P} ) .$$

Embodiment 19: The method according to the preceding embodiment, wherein the method comprises measuring signal values of independent control samples using the in vitro diagnostic medical device, such as the mass spectrometry device, transforming the measured signal values of the independent control samples into concentration values by applying the signal adjustment function g₁ and the leading calibration curve and comparing the concentration values to pre-assigned target concentration values of the independent control samples, wherein the target concentration values cₖⱼₗ^{lead} are accepted if a deviation between the concentration values of the independent control samples and the pre-assigned target concentration values of the independent control samples is within a predefined tolerance range.

Embodiment 20: The method according to any one of the two preceding embodiments, wherein the method comprises at least one step of adjustment of the leading calibration curve using the at least one leading calibrator k, wherein the step of adjustment of the leading calibration curve comprises determining a second signal adjustment function g₂(sₗ^{theo}, q₁, . . . , q_{Q}) with q₁, . . . , q_{Q} being a set of parameters of the second signal adjustment function and Q≥1, by
measuring signal values sₖⱼₗ^{lead,meas} of the at least one leading calibrator k using a manufacturer's standing measurement procedure on the in vitro diagnostic medical device, such as the mass spectrometry device, with j denoting the instrument and/or hardware part j=1, . . . , J and J≥1, and l denoting the repeat l=1, . . . , L and L≥1,
calculating the theoretical signals sₖ^{theo} by

$${s}_{k}^{theo} = f ( {c}_{k j l}^{lead} , {\hat{p}}_{1} , … , {\hat{p}}_{P} ) , and$$

fitting the signal adjustment function g₂ thereby determining the fitted parameters \hat{q}₁, . . . , \hat{q}_{Q}.

Embodiment 21: The method according to the preceding embodiment, wherein the method comprises at least one step of target value assignment of product calibrators comprising assigning at least one target concentration value cₖⱼₗ^{pr} of at least one product calibrator k, wherein the assigning of the target concentration value cₖⱼₗ^{pr} of the product calibrator comprises measuring signal values sₖⱼₗ^{meas} of the product calibrator k with k=1, . . . , K, and K≥1 using the in vitro diagnostic medical device, such as the mass spectrometry device, with j denoting the instrument and/or hardware part j=1, . . . , J and J≥1, and/denoting the repeat l=1, . . . , L and L≥1, wherein the assigning of the target concentration value cₖⱼₗ^{pr} of the product calibrator further comprises transforming the measured signal values sₖⱼₗ^{pr,meas}, into target concentration values cₖⱼₗ^{pr} by applying the following inverse functions consecutively:

$${s}_{kjl}^{pr , theo} = {g}_{2}^{- 1} ( {s}_{kjl}^{pr , meas} , {( {\hat{q}}_{1} , … , {\hat{q}}_{Q} )}_{j} ) , and$$

$${c}_{kjl}^{pr} = {f}^{- 1} ( {s}_{kjl}^{pr , theo} , {\hat{p}}_{1} , … , {\hat{p}}_{P} ) .$$

Embodiment 22: The method according to the preceding embodiment, wherein the method comprises measuring signal values of independent control samples using the in vitro diagnostic medical device, such as the mass spectrometry device, transforming the measured signal values of the independent control samples into concentration values by applying the signal adjustment function g₂ and the leading calibration curve and comparing the concentration values to pre-assigned target concentration values of the independent control samples, wherein the target concentration values cₖⱼₗ^{pr} are accepted if a deviation between the concentration values of the independent control samples and the pre-assigned target concentration values of the independent control samples is within a predefined tolerance range.

Embodiment 23: The method according to any one of the preceding embodiments, wherein the method further comprises performing at least one customer-side calibration step.

Embodiment 24: A method for establishing metrological traceability for at least one in vitro diagnostic medical device (110), wherein the method comprises a sequence of calibration steps and adjustment steps, wherein an outcome of each step depends on the outcome of the previous step,
wherein the method comprises providing a leading calibration curve, wherein the leading calibration curve fₚ describes a relationship of at least one concentration c of at least one analyte in at least one sample with a signal s of the sample measured with the in vitro diagnostic medical device (110), wherein the leading calibration curve fₚ is a parametrized function fₚ(c, \hat{p}₁, . . . , \hat{p}_{P}) with parameters \hat{p}₁, . . . , \hat{p}_{P} being a set of parameters of the leading calibration curve and P≥1,
wherein in each adjustment step a concentration adjustment function hₛ with s being a set of parameters of the concentration adjustment function, describing a relationship between a theoretical concentration value of measured signal values of first calibrator samples and a pre-assigned target concentration value of the first calibrator samples, is determined, wherein the theoretical concentration value of the first calibrator samples is determined by applying the inverse of the leading calibration curve f_{P}⁻¹ using the measured signal values of the first calibrator samples,
wherein the assignment of the target concentration value comprises applying the inverse of the leading calibration curve f_{P}⁻¹ using measured signal values of second calibrator samples, thereby obtaining theoretical concentration values, and applying the concentration adjustment function hₛ⁻ or the inverse of the concentration adjustment function hₛ⁻¹ on said theoretical concentration values, thereby obtaining the target concentration values of the second calibrator samples.

Embodiment 25: The method according to the preceding embodiment, wherein the concentration adjustment function connects pre-assigned target concentration values of the first calibrator samples with the theoretical concentration values of the measured signal of the first calibrator samples and the assignment of the target concentration values of the second calibrator samples is determined by applying the concentration adjustment function.

Embodiment 26: Processing device (112) wherein the processing device is configured for retrieving and/or storing at least one pre-determined leading calibration curve fₚ, wherein the processing device is further configured for storing a set of parameters \hat{p}₁, . . . , \hat{p}_{P} of the leading calibration curve fₚ, wherein P is a positive integer, wherein the leading calibration curve fₚ describes a relationship of at least one concentration c of at least one analyte in at least one sample with a signal s of the sample measured with an in vitro diagnostic medical device (110), wherein the leading calibration curve fₚ is a parametrized function fₚ(c, \hat{p}₁, . . . , \hat{p}_{P}), wherein the processing device (112) is further configured for performing a method for establishing metrological traceability for at least one in vitro diagnostic medical device (110) according to any one of the preceding embodiments referring to a method.

Embodiment 27: The processing device (112) according to the preceding embodiment, wherein the processing device (112) is an element of an in vitro diagnostics medical device (110) or a further device.

Embodiment 28: A kit comprising an in vitro diagnostic medical device (110), and a set of product calibrators and their target concentration values, wherein the target concentration values of said product calibrators are assigned by using a method according to any one of the preceding embodiments 1 to 23 or 24 to 25.

Embodiment 29: A computer program, wherein the computer program is adapted to perform a method according to any one of the preceding embodiments 1 to 23 or 24 to 25 while the program is being executed on a computer.

Embodiment 30: A computer program product having program code means, wherein the program code means can be stored or are stored on a storage medium, for performing the method according to any one of the preceding embodiments 1 to 23 or 24 to 25 when the program code means are executed on a computer or on a computer network.

### SHORT DESCRIPTION OF THE FIGURES

Further optional features and embodiments will be disclosed in more detail in the subsequent description of embodiments, preferably in conjunction with the dependent claims. Therein, the respective optional features may be realized in an isolated fashion as well as in any arbitrary feasible combination, as the skilled person will realize. The scope of the invention is not restricted by the preferred embodiments. The embodiments are schematically depicted in the Figures. Therein, identical reference numbers in these Figures refer to identical or functionally comparable elements.

In the Figures:

FIG. 1 shows a schematic view of a processing device as an element of an in vitro diagnostic medical device;

FIG. 2 shows a flowchart of an exemplary embodiment of a method for establishing metrological traceability for at least one in vitro diagnostic medical device;

FIGS. 3A and 3B show a diagram of leading calibration curves per analyte (3A) and adjustments of the leading calibration curves (3B);

FIG. 4 shows a flowchart giving an overview of two methods for establishing metrological traceability for at least one in vitro diagnostic medical device; and

FIGS. 5 to 10D show an exemplary example of the method according to the present invention.

### DETAILED DESCRIPTION OF THE EMBODIMENTS

FIG. 1 shows a schematic view of a processing device 112, which forms part of an in vitro diagnostic medical device 110. The in vitro diagnostic medical device 110 may be a medical device, which is configured for in vitro examination of at least one sample derived from the human body. Additionally of alternatively, the in vitro diagnostic medical device 110 may be a medical device configured for providing information for diagnostic, monitoring or compatibility purposes. The in vitro diagnostic medical device 110 may be used alone or in combination with further devices. The in vitro diagnostic medical device 110 may comprise one or more of at least one reagent, at least one calibrator, at least one control material, at least one specimen receptacle, software, related instruments or apparatus or other articles. The in vitro diagnostic medical device 110 illustrated in FIG. 1 is embodied as a mass spectrometry device 111. Further possibilities are feasible.

The processing device 112 may be a device or a combination of devices configured for controlling at least one function of at least one other device, such as of at least one other component of the in vitro diagnostics medical device 110, e.g. one other component of the mass spectrometry device 111, as shown in FIG. 1. The processing device 112 may e.g. comprise at least one processor 118 and/or at least one data storage device 120. The processing device 112 may comprise a plurality of processors 118 and/or a plurality of storage devices 120. The in vitro diagnostic medical device 110 may comprise further processors 118 as illustrated in FIG. 1. The at least one processing device 112 may comprise at least one data processing device having a software code stored thereon comprising a number of computer commands. As depicted in FIG. 1, the processing device 112 may be an element of an in vitro diagnostics medical device 110. Additionally or alternatively, the processing device 112 may be an element of a further device, e.g. a remote device.

The processing device 112 is configured for retrieving and/or storing at least one pre-determined leading calibration curve fₚ, and a set of parameters \hat{p}¹, . . . , \hat{p}_{P} of the leading calibration curve fₚ, wherein P is a positive integer. The leading calibration curve fₚ describes a relationship of at least one concentration c of at least one analyte in at least one sample with a signal s of the sample measured with the in vitro diagnostic medical device 110, such as the mass spectrometry device 111. The leading calibration curve fₚ is a parametrized function fₚ(c, \hat{p}₁, . . . , \hat{p}_{P}). The leading calibration curve may be a mathematical function having a set of parameters \hat{p}₁, . . . , \hat{p}_{P}. The parameter may be a mathematical quantity, which influences an output or a behavior of a mathematical function, and which is viewed as being held constant. Thus, the set of parameters \hat{p}₁, . . . , \hat{p}_{P} of the leading calibration curve fₚ may be configured for determining the behavior of the leading calibration curve. The leading calibration curve may comprise at least one mathematical operation, e.g. a multiplication with at least one factor or any other type of mathematical operation. The leading calibration curve may be formed, for example determined, by a functional form of the leading calibration curve together with fitted parameters values \hat{p}₁, . . . , \hat{p}_{P}. As an example, the functional form may be one or more of a Rodbart model function, a Padé model function, a quadratic model function, or any other non-linear or linear function. For example, Padé model function may be given by

$$y = \frac{{p}_{1} + {p}_{2} \cdot x}{1 + {p}_{3} \cdot x} ,$$

where y is signal value, x is concentration or target value and p₁, p₂ and p₃ are the parameters of the function. For example, Rodbard model function is given by

$$y = \frac{{p}_{1} - {p}_{4}}{1 + {( \frac{x}{{p}_{2}} )}^{{p}_{3}}} + {p}_{4} ,$$

where y is signal value, x is concentration or target value and p₁, p₂, p₃ and p₄ are the parameters of the function. Other functional forms are also feasible.

The leading calibration curve may be an assay and/or application-specific function. For example, multiple conditions may be used for its determination, e.g. one or more of different instruments, different reagent lots and different measurement conditions that may be used in the assay or application. The leading calibration curve may be provided e.g. by using multiple conditions such as one or more of multiple instruments, reagent lots.

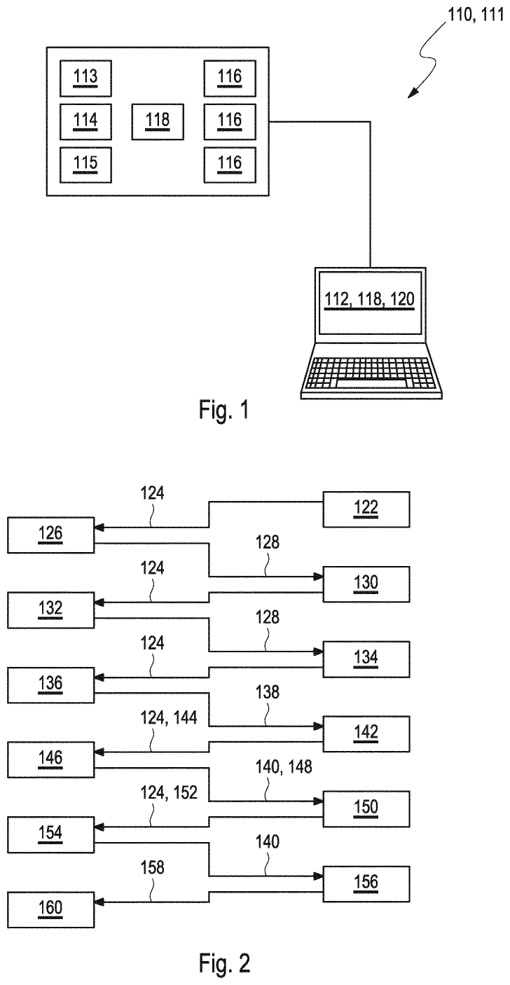

The processing device 112 is further configured for performing one or both of the methods for establishing metrological traceability for at least one in vitro diagnostic medical device 110 according to any one of the embodiments disclosed herein. Examples and/or details of the methods for establishing metrological traceability for at least one in vitro diagnostic medical device 110 will be discussed further below, e.g. with respect to FIGS. 2, 3A, 3B and 4. The pre-determined leading calibration curve that may be stored by the processing device 112 was pre-determined on the manufacture's side by using one or both of the methods according to the present invention.

The mass spectrometer device 111 may be a mass analyzer configured for detecting at least one analyte in at least one sample based on a mass to charge ratio. The mass spectrometer device 111 may be or may comprise at least one quadrupole mass analyzer 113 comprising at least one quadrupole as mass filter configured for selecting ions injected to the mass filter according to their mass-to-charge ratio m/z. The mass filter may comprise two pairs of electrodes. The electrodes may be rod-shaped, e.g. cylindrical. In an ideal case, the electrodes may be hyperbolic. The electrodes may be designed identically. The electrodes may be arranged in parallel extending along a common axis, e.g. a z axis. The quadrupole mass analyzer 113 may comprise a plurality of quadrupoles. For example, the quadrupole mass analyzer 113 may be a triple quadrupole mass spectrometer. The quadrupole mass analyzer 113 may comprise at least one power supply circuitry configured for applying at least one direct current (DC) voltage and at least one alternating current (AC) voltage between the two pairs of electrodes of the mass filter. The power supply circuitry may be configured for holding each opposing electrode pair at identical potential. The power supply circuitry may be configured for changing sign of charge of the electrode pairs periodically such that stable trajectories are only possible for ions within a certain mass-to-charge ratio m/z. Trajectories of ions within the mass filter can be described by the Mathieu differential equations. For measuring ions of different m/z values DC and AC voltage may be changed in time such that ions with different m/z values can be transmitted to a detector 114 of the mass spectrometry device 111 as illustrated in FIG. 1.

As shown in FIG. 1, the mass spectrometry device 111 may further comprise at least one ionization source 115 configured for generating ions, e.g. from neutral gas molecules. The ionization source 115 may be or may comprise at least one source selected from the group consisting of: at least one gas phase ionization source such as at least one electron impact (EI) source or at least one chemical ionization (CI) source; at least one desorption ionization source such as at least one plasma desorption (PDMS) source, at least one fast atom bombardment (FAB) source, at least one secondary ion mass spectrometry (SIMS) source, at least one laser desorption (LDMS) source, and at least one matrix assisted laser desorption (MALDI) source; at least one spray ionization source such as at least one thermospray (TSP) source, at least one atmospheric pressure chemical ionization (APCI) source, at least one electrospray (ESI), and at least one atmospheric pressure ionization (API) source.

The detector 114 of the mass spectrometry device 111 may be configured for detecting incoming ions such as charged particles. The detector 114 may be or may comprise at least one electron multiplier. The mass spectrometry device 111, e.g. the detector 114 and/or at least one processing unit 118 of the mass spectrometry device 111, which may also be referred to as processor 118, may be configured to determine at least one mass spectrum of the detected ions, e.g. a two dimensional representation of signal intensity vs the charge-to-mass ratio m/z, wherein the signal intensity corresponds to abundance of the respective ion. The mass spectrum may be a pixelated image. For determining resulting intensities of pixels of the mass spectrum, signals detected with the detector 114 within a certain m/z range may be integrated. The analyte in the sample may be identified by the processing unit 118. The processing unit 118 may be configured for correlating known masses to the identified masses or through a characteristic fragmentation pattern.

The mass spectrometry device 111 may be or may comprise a liquid chromatography mass spectrometry device. The mass spectrometry device 111 may be connected to and/or may comprise at least one liquid chromatograph (not shown), which may be used for sample preparation for the mass spectrometry device 111. Other embodiments of sample preparation may be possible, such as at least one gas chromatograph.

The sample may, e.g. be a solid, liquid, or gaseous sample. As an example, the sample may be a biological sample, e.g. a human sample or a pool of human samples. For example, the sample may be a liquid sample, e.g. an aqueous sample. For example, the test sample may be selected from the group consisting of: a physiological fluid, including whole blood, serum, plasma, saliva, ocular lens fluid, lacrimal fluid, cerebrospinal fluid, sweat, urine, milk, ascites, mucus, synovial fluid, peritoneal fluid, and amniotic fluid; lavage fluid; tissue, cells, or the like. The sample may, however, also be a natural or industrial liquid, e.g. surface or ground water, sewage, industrial wastewater, processing fluid, soil eluates, and the like. The sample may comprise one or more further chemical compounds, which are not to be determined and which are commonly referred to as matrix. The sample may be used directly as obtained from the respective source or may be subjected to one or more pretreatment and/or a sample preparation step(s). Thus, the sample may be pretreated by physical and/or chemical methods, for example by centrifugation, filtration, mixing, homogenization, chromatography, precipitation, dilution, concentration, contacting with a binding and/or detection reagent, and/or any other method deemed appropriate by the skilled person. In, i.e. before, during, and/or after, the sample preparation step, one or more internal standard(s) may be added to the sample. The sample may be spiked with the internal standard. For example, an internal standard may be added to the sample at a predefined concentration. The internal standard may be selected such that it is easily identifiable under normal operating conditions of the detector chosen, e.g. a mass spectrometry device, a photometric cell, e.g. in an UV-Vis spectroscopic device, an evaporative light scattering refractometer, a conductometer, or any device deemed appropriate by the skilled person. The concentration of the internal standard may be pre-determined and significantly higher than the concentration of the analyte.

The sample may comprise one or more analytes of interest. The analyte may e.g. be a chemical, biochemical or biological compound, e.g. a molecule or a fragment thereof, detected by the mass spectrometry device 111 during the measurement of the sample. As a result of the measurement process, the mass spectrometry device may detect a presence and/or an abundance and/or a concentration of one or more analytes, e.g. a plurality of analytes, in the sample. The analyte may be a sample component as such. Additionally or alternatively, the analyte may be a fragment of a component present in the sample. As an example, one or more of the sample components may be fragmented during the measurement process, e.g. during an ionization procedure, such that a single sample component may yield a plurality of different fragments, e.g. charged fragments, which may at least partially be detected as analytes by the mass spectrometry device.

The concentration c of the analyte may, for example, be determined as an abundance of the analyte in a given volume set in relation to said volume, such as the sample volume. The concentration may e.g. be described by at least one of: a mass concentration, a molar concentration, and a volume concentration. The concentration of the analyte may be specified and/or quantified by a concentration value.

The in vitro diagnostic medical device 110, such as the mass spectrometry device 111, may comprise a plurality of hardware parts 116. The hardware part 116 may be a physical and/or tangible part of the in vitro diagnostic medical device 110. The hardware part 116 may e.g. comprise an instrument or a component of an instrument that forms part of the in vitro diagnostic medical device 110, such as of the mass spectrometry device 111. E.g. the hardware part 116 may be part of one or more of: a sample preparation unit of the mass spectrometry device 111, an ionization unit of the mass spectrometry device 111, a mass analyzer unit of the mass spectrometry device 111 and a detection unit of the mass spectrometry device 111. For example, the hardware part 116 may be part of at least one of: the quadrupole mass analyzer 113, the detector 114, the ionization source 115. Further possibilities are feasible. The hardware part 116 may have a specific configuration or setting that may be variable or adjustable, e.g. in an application-specific manner. Additionally or alternatively, the configuration or the setting may vary due to manufacturing tolerances. As an example, due to the potential variability of the hardware part 116, a calibration of the hardware part 116 may be required, e.g. a calibration comprising one or more calibration steps, such as those forming part of the sequence of calibration and adjustment steps of one or both of the methods for establishing metrological traceability for at least one in vitro diagnostic medical device 110.

In FIG. 2, an exemplary embodiment of a method for establishing metrological traceability for at least one in vitro diagnostic medical device 110 is shown in a schematic flowchart. The method comprises a sequence of calibration and adjustment steps, wherein an outcome of each step depends on the outcome of the previous step. The method may further comprise additional method steps, which are not listed. Further, one or more or even all of the method steps, may be performed only once or repeatedly, e.g. to produce at least one averaged value. As an example, a target concentration value assigned as part of the method may be assigned as at least one averaged value.

In the following the method for establishing metrological traceability for at least one in vitro diagnostic medical device 110 will be described by means of the specific embodiment illustrated in FIG. 2. In the specific embodiment described, the in vitro diagnostic medical device 110 is a mass spectrometry device 111. It is readily understood by those skilled in the art that, in general, the method may also be carried out using other in vitro diagnostic medical devices 110.

FIG. 2 illustrates materials, which are used during the method, e.g. samples, in boxes on the left hand side. Measurement procedure, e.g. for sample measurements, are illustrated in boxes on the right hand side.

FIG. 2 shows an exemplary sequence of calibration and adjustment steps comprising a plurality of calibration and/or adjustment steps. The method may comprise a whole standardization procedure. An outcome of each step depends on the outcome of the previous step. The method may comprise a hierarchy of calibration and adjustment steps. The sequence of calibration and adjustment steps may comprise performing method steps from a reference to the final measuring system, where the outcome of each step depends on the outcome of the previous step. The method may comprise establishing metrological traceability by ensuring traceability to higher order reference system components as required by ISO 17511:2020. The metrological traceability may refer to the hierarchy of calibration and adjustment steps and a sequence of value assignments, which may allow an unbroken linkage between a measurement result for the sample up to the highest available reference system component in the hierarchy.

As depicted in FIG. 2, the sequence of calibration and adjustment steps may comprise a first calibration and adjustment step using a fit for purpose measurement procedure for purity assessment e.g. quantitative NMR, mass balance or the like (“p.1. Fit for purpose measurement procedures for purity assessment”), illustrated by reference sign 122. A target concentration value for at least one certified primary reference material (“m.1. certified primary reference material (CRM)”), denoted with reference sign 126, may be assigned by the first calibration and adjustment step, denoted with reference sign 128.

The sequence of calibration and adjustment steps may further comprise a second calibration and adjustment step using a primary reference measurement procedure for calibrator preparation, denoted with reference sign 130, e.g. gravimetric preparation, (“p.2. primary reference measurement procedure for calibrator preparation”) on the at least one certified primary reference material 126. The primary reference measurement procedure 130 may be or may comprise a reference measurement procedure used to obtain a measurement result without relation to a measurement standard for a quantity of the same kind. The CRM 126 may be a reference material accompanied by documentation issued by an authoritative body and providing one or more specified property values with associated uncertainties and traceabilities, using valid procedures. The primary reference measurement procedure for calibrator preparation 130 and the CRM 126 may fulfill the requirements described in ISO 17511:2020 and ISO 15194.

The sequence of calibration and adjustment steps may further comprise a third calibration and adjustment step using a primary reference measurement procedure for a measurand (“p.3 primary reference measurement procedure for the measurand”), denoted with reference sign 134, on at least one primary calibrator (“m.2. primary calibrator prepared as solution of m.1 in suitable solvent”), denoted with reference sign 132. The measurand may be a quantity intended to be measured. The calibrator may be material used as measurement standard. The primary calibrator 132 may be a measurement standard established using a primary reference measurement procedure, or created as an artefact, chosen by convention. The primary calibrator 132 may be prepared as solution of the CRM 126 in a suitable solvent. The primary reference measurement procedure for a measurand and the primary calibrator 132 may fulfill the requirements described in ISO 17511:2020. The target concentration value for the primary calibrator 132 may be assigned by the second calibration and adjustment step 130.

The sequence of calibration and adjustment steps may further comprise a forth calibration and adjustment step using a manufacturer selected measurement procedure (“p4. manufacturer selected measurement procedure”), denoted with reference number 142, on at least one secondary calibrator (“m.3 secondary calibrator (reference samples or pools or human samples)”), denoted with reference sign 136. The secondary calibrator 136 may be a measurement standard established through calibration with respect to a primary measurement standard for a quantity of the same kind. The manufacturer selected measurement procedure 142 and the secondary calibrator 136 may fulfill the requirements described in ISO 17511:2020.

The secondary calibrator 136 may be at least one of: human samples, pools of human samples, samples with matrix and samples comparable to human samples. The manufacturer's selected measurement procedure 142 may comprise one or more of homogenous or heterogeneous immunoassays or liquid chromatography coupled with tandem mass spectrometry (LC-MS/MS). Other measurement procedures are feasible. The manufacturer's selected measurement procedure 142 may be at least partially automated. The manufacturer's selected measurement procedure 142 may for example be comparable to or attuned to a customer's measurement procedure. The target concentration value for the secondary calibrator 136 may be assigned by the third calibration and adjustment step 134.

The sequence of calibration and adjustment steps may further comprise a fifth calibration and adjustment step using a manufacturer's standing measurement procedure (“p5. manufacturer's standing measurement procedure”), denoted with reference sign 150, on at least one leading calibrator (“m.4. manufacturer's working calibrator (leading calibrator)”), denoted reference sign 146 in FIG. 2. The leading calibrators may correspond to the samples denoted as manufacturer's working calibrator in the standard ISO 17511:2020. The target concentration value for the leading calibrator 146 may be assigned by the fourth calibration and adjustment step 142.

The sequence of calibration and adjustment steps may further comprise a sixth calibration and adjustment step using a measurement procedure on the end user's IVD medical device 110 (“p.6. end-users IVD MD”), denoted with reference sign 156, on at least one product calibrator (“m.5. end user IVD MD calibrator (product calibrator)”), denoted with reference sign 154 in FIG. 2. The target concentration value for the product calibrator 154 may be assigned by the fifth calibration and adjustment step 150. The sixth calibration and adjustment step 156 may further comprise assigning target concentration values to human samples (“m.6. human samples with results”), denoted with reference sign 160 in FIG. 2.

The method comprises providing a leading calibration curve. The leading calibration curve may be an arbitrary mathematical function describing a relationship of at least one concentration c of at least one analyte in at least one sample with a signal s of the sample measured with the mass spectrometry device. The leading calibration curve, in particular the inverse leading calibration curve, may assign a concentration c to a sample examined by the in vitro diagnostic medical device on the basis of at least one of: a measured signal value and a theoretical signal value derived from the measured signal value. Additionally or alternatively, the leading calibration curve may contribute to assigning a concentration c to a sample, by assigning a theoretical concentration value to a sample on the basis of the measured signal value of the sample, wherein the concentration c is assigned to the sample on the basis of the theoretical concentration value in a further step, e.g. by applying a concentration adjustment function or its inverse.

The leading calibration curve may comprise at least one mathematical operation, e.g. a multiplication with at least one factor or any other type of mathematical operation. The leading calibration curve is a parametrized function. The leading calibration curve fₚ is a parametrized function fₚ(c, \hat{p}₁, . . . , \hat{p}_{P}) with parameters \hat{p}₁, . . . , \hat{p}_{P} being a set of parameters of the leading calibration curve and P≥1. The leading calibration curve may be formed, for example determined, by a functional form of the leading calibration curve together with fitted parameters values \hat{p}₁, . . . , \hat{p}_{P}. The functional form may e.g. be one or more of a Rodbart model function, a Padé model function, a quadratic model function, or any other non-linear or linear function. For example, Padé model function may be given by

$$y = \frac{{p}_{1} + {p}_{2} \cdot x}{1 + {p}_{3} \cdot x} ,$$

where y is signal value, x is concentration or target value and p₁, p₂ and p₃ are the parameters of the function. For example, Rodbard model function is given by

$$y = \frac{{p}_{1} - {p}_{4}}{1 + {( \frac{x}{{p}_{2}} )}^{{p}_{3}}} + {p}_{4} ,$$

where y is signal value, x is concentration or target value and p₁, p₂, p₃ and p₄ are the parameters of the function. Other functional forms are also feasible.

The providing of the leading calibration curve may comprise one or more of determining the leading calibration curve by at least one of setting and choosing the leading calibration curve. For example, the process of setting the leading calibration curve may comprise establishing the leading calibration curve, e.g. by determining at least one of its form and/or at least one of the parameter values \hat{p}₁, . . . , \hat{p}_{P}, such as by choosing at least one model function and/or by fitting at least one of the parameter values. The setting of the leading calibration curve may, for example, comprise generating at least one specific parameter value in a modelling and/or fitting procedure.

The leading calibration curve may be an assay and/or application-specific function. For example, multiple conditions may be used for its determination, e.g. one or more of different instruments, different reagent lots and different measurement conditions that may be used in the assay or application. For example, the leading calibration curve may be determined by using multiple conditions such as one or more of multiple instruments, and/or hardware parts and/or reagent lots and the like.

The providing of the leading calibration curve may be part of at least one of the calibration and adjustment steps. The providing of the leading calibration curve may be performed at a high order step of the hierarchy, e.g. one of the first steps in the hierarchy. For example, the leading calibration curve may be determined by using at least one primary calibrator 132. At least one target concentration value of the primary calibrator 132 may be established based on a primary reference measurement procedure for a calibrator preparation 130. For example, the leading calibration curve is provided by using at least one secondary calibrator 136. At least one target concentration value of the secondary calibrator 136 may be established based on the primary reference measurement procedure for a measurand 134. Other examples for providing of the leading calibration function in other steps of the hierarchy are also feasible. Additionally or alternatively, the leading calibration curve may be provided by retrieving a leading calibration curve such as from at least one database, e.g. a cloud. The providing of the leading calibration curve may be performed once or repeatedly.

For example, the step of providing the leading calibration curve may be embodied as comprising the setting of the leading calibration curve. For example, this step may be performed as one of the initial steps of the standardization process. The step of setting of the leading calibration curve may comprise the determining of the functional form of the leading calibration curve. The step of setting of the leading calibration curve may comprise the determining of the parameter values of the parameters \hat{p}₁, . . . , \hat{p}_{P} of the leading calibration curve fₚ(c, \hat{p}₁, . . . , \hat{p}_{P}).

As shown in FIG. 2, the method may comprise at least one step of setting of the leading calibration curve. The step of “Setting of the leading calibration curve” is marked with reference sign 138 in FIG. 2. In the embodiment of FIG. 2, said step of setting of the leading calibration curve may comprise measuring signal values sᵢⱼₗ^{meas} of a set of secondary calibrators 136 with the in vitro diagnostic medical device 110, with i denoting the secondary calibrators i=1, . . . , I and I≥1, j denoting the instrument and/or hardware part j=1, . . . , J and J≥1, and/denoting the repeat l=1, . . . , L and L≥1, and determining parameter values of the parameters \hat{p}₁, . . . , \hat{p}_{P} of the leading calibration curve fₚ(c, \hat{p}₁, . . . , \hat{p}_{P}) using pre-assigned target concentration values cᵢ of the set of secondary calibrators and the measured signal values sᵢⱼₗ^{meas}. The set of secondary calibrator 136 may comprise at least one secondary calibrator 136, such as at least three secondary calibrators 136, e.g. 20 to 30 secondary calibrators 136. The set of secondary calibrators 136 may, however also comprise a different number of secondary calibrators 136, e.g. more than 30 secondary calibrators 136. The target concentration values cᵢ of the secondary calibrators 136 may be pre-assigned in a higher order calibration and/or adjustment step, e.g. comprising a primary reference measurement procedure for a measurand 134.

A relationship between the measured signal values of calibrator samples and the target concentration values of said calibrator samples may be described only insufficiently, such as not precisely or accurately enough, by the leading calibration curve. As an example, the concentration values as determined using the leading calibration curve on the basis of the measured signal values of said calibrator samples may differ from the target concentration values of said calibrator samples such as by more than a pre-determined threshold value. For example, this may result from the fact that the leading calibration curve was determined on the basis of samples other than said calibrator samples. In the standardization process shown in FIG. 2, the leading calibration curve may nevertheless be kept unchanged and, instead, a further function, the signal adjustment function may be determined and/or adjusted. While the standardization process comprises several calibration and adjustment steps, the leading calibration curve may be established initially, such as in a high order step of the hierarchy, and may be kept unchanged over the two or more subsequent adjustment steps.

In each adjustment step, a signal adjustment function gᵣ with r being a set of parameters of the signal adjustment function, describing a relationship between measured and theoretical signal values, is determined by determining the relationship between measured signal values of first calibrator samples and theoretical signal values of the first calibrator samples derived from the leading calibration curve. The theoretical signal values are determined by applying the leading calibration curve using pre-assigned target concentration values cᵢ of the first calibrator samples. Each adjustment step comprises assigning at least one target concentration value from measured signal values of at least one second calibrator sample. The assigning comprises determining at least one theoretical signal value of the second calibrator sample by applying the signal adjustment function determined in the previous adjustment step or the inverse of the signal adjustment function determined in the previous adjustment step to the measured signal values of the second calibrator sample and applying the inverse leading calibration curve f_{P}⁻¹ to the theoretical signal value of the second calibrator sample.

The method comprises a plurality of successively performed calibration and adjustment steps as outlined above. For example, as shown in FIG. 2, the method may comprise setting of the leading calibration curve 138; adjustment of the leading calibration curve 140; target value assignment of leading calibrators 144; adjustment of the leading calibration curve by leading calibrators 148; target value assignment of product calibrators 152. The steps may be performed at the manufacturer's side.

Subsequently to the setting of the leading calibration curve 138, as shown in FIG. 2, the method may further comprise at least one step of adjustment of the leading calibration curve 140 comprising determining a first signal adjustment function g₁(sᵢ^{theo}, r₁, . . . , r_{R}) using a set of secondary calibrators i=1, . . . . I, I≥1, wherein the determining of the first signal adjustment function comprises
measuring signal values sᵢⱼₗ^{meas} of the secondary calibrators 136 using a manufacturer's selected measurement procedure 142,
calculating the theoretical signals sᵢ^{theo} by

$${s}_{i}^{theo} = f ( {c}_{i} , {\hat{p}}_{1} , … , {\hat{p}}_{P} ) ,$$

with cᵢ being the pre-determined target concentration values of the secondary calibrators 136, and
fitting the signal adjustment function g₁ thereby determining the fitted parameters \hat{r}₁, . . . , \hat{r}_{R}.

The step of adjustment of the leading calibration curve 140 may, for example, comprise determining the first signal adjustment function, while keeping the leading calibration curve unchanged. The pre-assigned target concentration values of the secondary calibrators 136 may be determined in a higher order calibration and/or adjustment step, e.g. comprising a primary reference measurement procedure for a measurand 134.

In the subsequent calibration and adjustment step the two functions, i.e. the leading calibration function and the first signal adjustment function, may be used in conjunction to assign at least one target concentration value to a sample, e.g. the leading calibrators 146 in the step of target value assignment of leading calibrators 144. Additionally or alternatively, the target concentration value may be assigned to a sample on the basis of the leading calibration curve alone, e.g. by applying the inverse of the leading calibration curve to the measured signal values of the sample as measured using the in vitro diagnostic medical device 110. For example, the target concentration value may be assigned to the sample on the basis of the leading calibration curve alone in case the assignment of the target concentration value is performed before the first signal adjustment function was determined. As an example, the step of target value assignment of leading calibrators 144 may initially be carried out in direct subsequence to the step of setting of the leading calibration curve 138. In this case, no signal adjustment function may have been determined yet and thus, no signal adjustment function may be available. The target concentration values may be assigned to the leading calibrators using the leading calibration curve alone, e.g. by applying the inverse of the leading calibration curve to the measured signal values of the leading calibrator. Alternatively, the step of target value assignment of leading calibrators 144 may be carried out in a repetition of steps, such that the step of adjustment of the leading calibration curve 140, which in the hierarchy of calibration and adjustment steps succeeds the step of target value assignment of leading calibrators 144, has already been carried out. Thus, for the repetition of step 144, the signal adjustment function may be available. In this case, the step of target values assignment of leading calibrators may be carried out using both the leading calibration curve and the signal adjustment function.

For example, the step of target value assignment of leading calibrators 144 may comprise assigning at least one target concentration value cₖⱼₗ^{lead} of at least one leading calibrator 146. The assigning of the target concentration value cₖⱼₗ^{lead} of the leading calibrator 146 may comprise measuring signal values sₖⱼₗ^{lead,meas} of the leading calibrator k with k=1, . . . , K, and K≥1 using the in vitro diagnostic medical device 110, with j denoting the instrument and/or hardware part j=1, . . . , J and J≥1, and l denoting the repeat l=1, . . . , L and L≥1. The assigning of the target concentration value cₖⱼₗ^{lead} of the leading calibrator 146 may further comprise transforming the measured signal values sₖⱼₗ^{lead,meas}, into target concentration values cₖⱼₗ^{lead} by applying the following inverse functions consecutively:

$${s}_{kjl}^{lead , theo} = {g}_{1}^{- 1} ( {s}_{kjl}^{lead , meas} , {( {\hat{r}}_{1} , … , {\hat{r}}_{R} )}_{j} ) ; and$$

$${c}_{kjl}^{lead} = {f}^{- 1} ( {s}_{kjl}^{lead , theo} , {\hat{p}}_{1} , … , {\hat{p}}_{P} ) .$$

The target concentration value of the leading calibrator 146 may e.g. be assigned as at least one averaged value from a plurality of target concentration values, wherein the average value is one or more of mean, median or weighted mean value of the plurality of target concentration values.

As shown in FIG. 2, the step of “Adjustment of the leading calibration curve” 140 may e.g. be embodied as a step of “Adjustment of the leading calibration curve by leading calibrators” 148 using the at least one leading calibrator k. The step of adjustment of the leading calibration curve by leading calibrators 146 may comprise determining a second signal adjustment function g₂(sₖ^{theo}, q₁, . . . , q_{Q}) with q₁, . . . , q_{Q} being a set of parameters of the second signal adjustment function and Q≥1, by
measuring signal values sₖⱼₗ^{lead,meas} of the at least one leading calibrator k 146 using a manufacturer's standing measurement procedure 150 on the in vitro diagnostic medical device 110, with j denoting the instrument and/or hardware part j=1, . . . , J and J≥1, and/denoting the repeat l=1, . . . , L and L≥1,
calculating the theoretical signals sₖ^{theo} by

$${s}_{k}^{theo} = f ( {c}_{kjl}^{lead} , {\hat{p}}_{1} , … , {\hat{p}}_{P} ) , and$$

fitting the signal adjustment function g₂ thereby determining the fitted parameters \hat{q}₁, . . . , \hat{q}_{Q}.

The manufacturer's standing measurement procedure 150, which may be used to measure the signal values of the leading calibrators 146, may comprise e.g. at least one of homogenous immunoassays, heterogeneous immunoassays and liquid chromatography coupled with tandem mass spectrometry (LC-MS/MS). Other measurement procedures are feasible.

The step of adjustment of the leading calibration curve by leading calibrators 148 may, for example, comprise determining the signal adjustment function g₂ using the measured signal values of the leading calibrators 146, while keeping the leading calibration curve itself unchanged. In the following the two functions, i.e. the leading calibration function and the second signal adjustment function, may be used in conjunction to assign the at least one target concentration value of at least one further second calibrator sample, e.g. of product calibrators 154.

The method may further comprise the at least one step of “Target value assignment of product calibrators” 152 in FIG. 2. The step of target value assignment of product calibrators 152 may comprise assigning at least one target concentration value cₖⱼₗ^{pr} of at least one product calibrator k. The assigning of the target concentration value cₖⱼₗ^{pr} of the product calibrator comprises measuring signal values sₖⱼₗ^{spr,meas} of the product calibrator k with k=1, . . . , K, and K≥1 using the in vitro diagnostic medical device, with j denoting the instrument and/or hardware part j=1, . . . , J and J≥1, and l denoting the repeat l=1, . . . , L and L≥1, wherein the determining of the target concentration value cⱼₖₗ^{pr} of the product calibrator 154 further comprises transforming the measured signal values sₖⱼₗ^{pr,meas}, into target concentration values cₖⱼₗ^{pr} by applying the following inverse functions consecutively:

$${s}_{kjl}^{pr , theo} = {g}_{2}^{- 1} ( {s}_{kjl}^{pr , meas} , {( {\hat{q}}_{1} , … , {\hat{q}}_{Q} )}_{j} ) , and$$

$${c}_{kjl}^{pr} = {f}^{- 1} ( {s}_{kjl}^{pr , theo} , {\hat{p}}_{1} , … , {\hat{p}}_{P} ) .$$

The target concentration value of the product calibrator may e.g. be assigned as at least one averaged value from a plurality of target concentration values, wherein the average value is one or more of mean, median or weighted mean value of the plurality of target concentration values.

The method may further comprise measuring signal values of independent control samples using the in vitro diagnostic medical device 110, e.g. the mass spectrometry device 111, and transforming the measured signal values of the independent control samples into concentration values by applying the signal adjustment function g₂ and the leading calibration curve. The method may further comprise comparing the concentration values to pre-defined target concentration values of the independent control samples. The target concentration values coil may be accepted if a deviation between the concentration values of the independent control samples and the pre-defined target concentration values of the independent control samples is within a predefined tolerance range.

The step of target value assignment of product calibrators 152 may be the last step of the method for establishing metrological traceability carried out at the manufacturer's side. The method may further comprise performing at least one customer-side calibration step. For example, the product calibrators with accepted target concentration values cₖⱼₗ^{pr} may be used for the customer-side calibration step. As an example a further step of adjustment of the leading calibration curve may be performed using the product calibrators with assigned target concentration values. The processing device 112 may further be configured for conducting the at least one customer-side calibration step for the IVD medical device 110 on the at least one set of product calibrators 154. Thereby the signal adjustment function may be calibrated. The customer-side calibration step may comprise
measuring signal values sₖⱼₗ^{pr,meas} of the at least one product calibrator k using a customer-side measurement procedure on the in vitro diagnostic medical device 110, e.g. the mass spectrometry device 111, with j denoting the instrument and/or hardware part 116 j=1, . . . , J and J≥1, and/denoting the repeat l=1, . . . , L and L≥1,
calculating the theoretical signals sᵢ^{theo} by

$${s}_{k}^{theo} = f ( {c}_{kjl}^{pr} , {\hat{p}}_{1} , … , {\hat{p}}_{P} ) ,$$

with cₖⱼₗ^{pr} being the pre-determined target concentration values of the set of product calibrators 154
fitting the signal adjustment function thereby determining the fitted parameters of the signal adjustment function.

The target concentration values may have been pre-assigned on the manufacturer's side by using the method for establishing metrological traceability as described in any one of the embodiments described above, e.g. in a step of target value assignment of human samples 158.

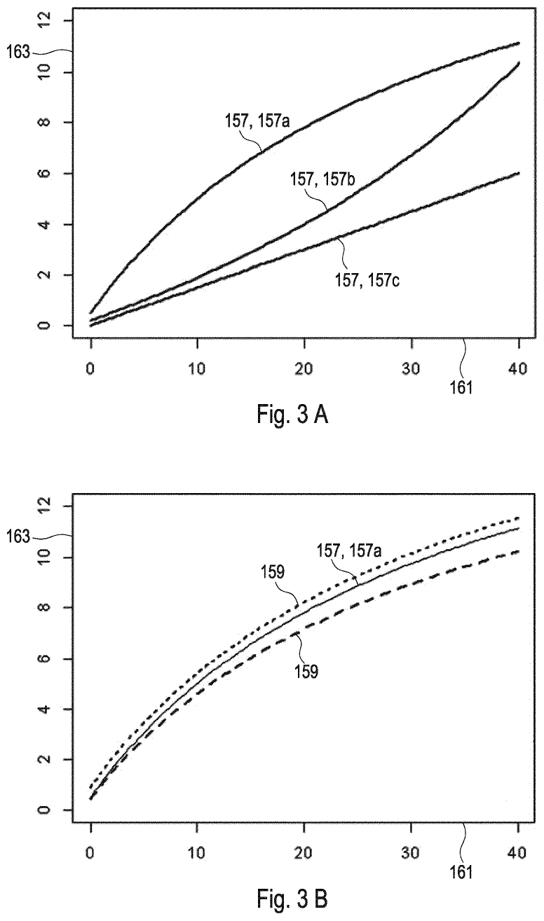

FIG. 3A shows a diagram of leading calibration curves 157 per analyte. Thus, FIG. 3A shows three leading calibration curves 157 pertaining to three different analytes. In FIG. 3A, the following curves are shown, one leading calibration curve marked as 157a for analyte A, one leading calibration curve 157b for analyte B and one leading calibration curve 157c for analyte C. The x-axis of the diagram shows the analyte concentration in arbitrary units 161. The y-axis of the diagram shows the signal in arbitrary units 162, e.g in a number of counts. FIG. 3B shows leading calibration curve 157a of FIG. 3A as a continuous line and two further curves 159 as dotted lines, which illustrate in an exemplary fashion the effect of two different signal adjustment functions on the relationship between the signal and the concentration of a particular analyte, in the given example Analyte A. Using and/or applying the signal adjustment function may correspond to the effect of tilting and/or shifting the leading calibration curve as illustrated in FIG. 3B.

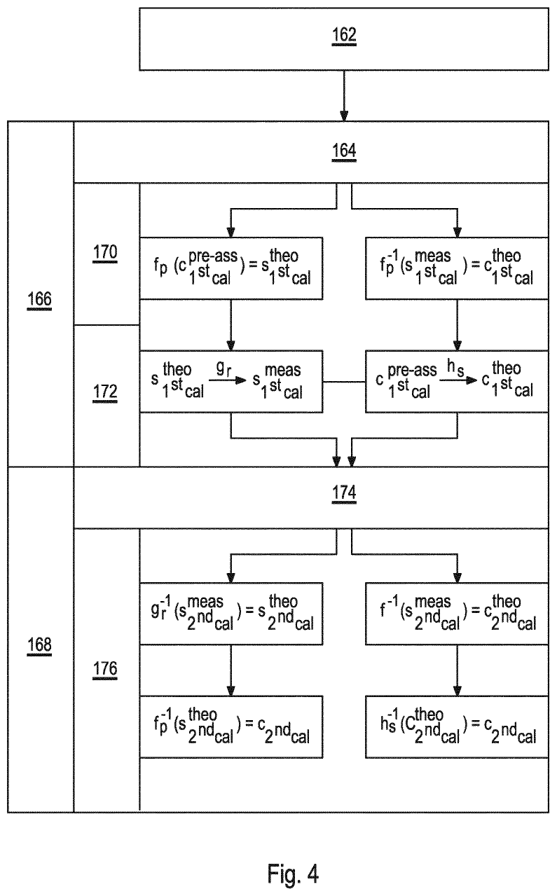

FIG. 4 shows a flowchart giving an overview of the two methods for establishing metrological traceability for at least one in vitro diagnostic medical device 110. Reference sign 162 in the top box indicates both methods as “methods for establishing metrological traceability for at least one in vitro diagnostic medical device”. Reference sign 164 in the following box indicates, what may be available at this point of both methods. Available may be the leading calibration curve, pre-assigned target concentration values of the first calibrator sample and measured signal values of the first calibrator sample. The leading calibration curve may in particular be abbreviated as “LCC”, the term” pre-assigned” may in particular be abbreviated as “pre-ass.”, and the term “first calibrator sample” may be abbreviated as “1^{st} cal”, specifically in FIG. 4 and its context. In the adjustment step, the first calibrator samples may be used as indicated in the lateral box marked 166. Subsequently, the second calibrator samples may be used, as indicated in the adjacent lateral box marked 168. In the adjustment step, first the leading calibration curve, or its inverse, may be applied as indicated in the box marked 170. As indicated in the respective box on the left hand side, the leading calibration curve may be applied to the pre-assigned target concentration values of the first calibrator sample to determine theoretical signal values of the first calibrator sample. Alternatively, and as indicated in parallel in the box on the right hand side, the inverse of the leading calibration curve may be applied to the measured signal of the first calibrator sample to determine a theoretical concentration of the first calibrator sample.

Next, the adjustment function may be determined as indicated in the box marked with reference sign 172. The signal adjustment function, which describes a relationship between measured and theoretical signal values, may be determined, as shown on the left hand side. In particular, the signal adjustment function may be fitted such that it maps the theoretical signal values of the first calibrator samples to the pre-assigned target concentration values of the first calibrator samples (this option is shown in FIG. 4) or vice versa (not shown). Alternatively and as shown in the right hand flow, the concentration adjustment function, which describes a relationship between the theoretical concentration of the first calibrator samples and the pre-assigned target concentration value of the first calibrator samples, may be determined. In particular, the concentration adjustment function may be fitted such that it maps the pre-assigned target concentration values of the first calibrator samples to the theoretical concentration values of the first calibrator samples (this option is shown in FIG. 4) or vice versa (not shown).

Next, the target concentration value may be assigned to the second calibrator sample as indicated in the box marked with reference sign 174. In particular, this may be done by consecutively applying the inverse of the adjustment function (or the adjustment function) and the inverse of the leading calibration curve or vice versa, as indicated by the lateral box marked 176. Thus, as shown in the flow on the left hand side, the inverse signal adjustment function may be applied to the measured signal values of the second calibrator samples to determine the theoretical signal values of the second calibrator samples and the inverse of the leading calibration curve may then be applied to the theoretical signal values of the second calibrator samples to assign the target concentration value to the second calibrator samples. It is noted that in case the above-mentioned option was used, in which the signal adjustment function was fitted such that it maps the pre-assigned target concentration values of the first calibrator samples to the theoretical signal values of the first calibrator samples (and not vice versa), the concentration adjustment function and not its inverse may be used. For a better overview, this option is again not shown in FIG. 4. The flow on the right hand side, shows the corresponding steps, in particular, the application of the inverse of the leading calibration curve to the measured signal values of the second calibrator samples to assign the theoretical concentration values to the second calibrator samples. This may be followed by applying the inverse of the concentration adjustment function to the theoretical concentration values of the second calibrator samples to assign the target concentration value to the second calibrator samples. Again it is noted, that in case the above-mentioned option was used, in which the concentration adjustment function was fitted such that it maps the theoretical concentration values of the first calibrator samples to the pre-assigned concentration values of the first calibrator samples, the concentration adjustment function and not its inverse may be used. For a better overview and comparison of the methods for establishing metrological traceability for at least one in vitro diagnostic medical device 110, this option is not illustrated in FIG. 4.

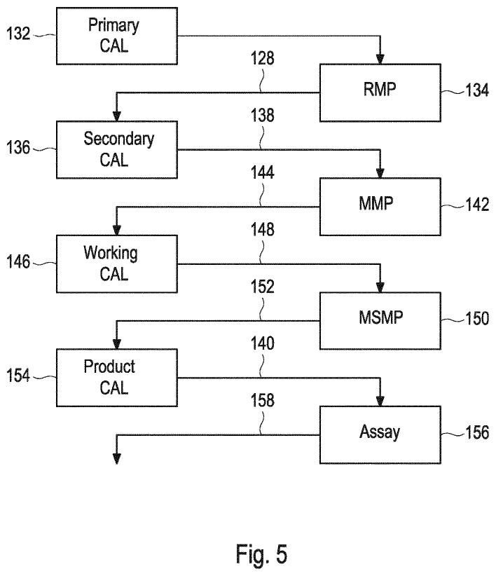

FIGS. 5 to 10 give an overview of an exemplary embodiment of the method for establishing metrological traceability for at least one in vitro diagnostic medical device 110.

FIG. 5 shows a flowchart of this exemplary embodiment.

In a first step, a reference measurement procedure for the measurand (“RMP”) 134 is performed on primary calibrators (“Primary CAL”) 132. Next, in calibration step 128, target values for secondary calibrators (“Secondary CAL”) 136 are determined using the RMP 134 on secondary calibrators 136.

In this exemplary embodiment, the leading calibration curve is set 138 by using the secondary calibrators 136. A manufacturer selected measurement procedure (“MMP”) 142 is performed on the secondary calibrators 136. This leading calibration curve is used in all subsequent steps, which is denoted with a curved bracket.

Next, in this embodiment, still in step 138, an adjustment of the leading calibration curve may be performed using the secondary calibrators 136, a manufacturer's selected measurement procedure (“MMP”) 142 and the assigned target concentration values.

Next, in step 144, the target concentration value for working calibrators (“Working CAL”) 146, also denoted as leading calibrators, are assigned using the manufacturer selected measurement procedure (“MMP”) 142.

Next, in step 148, an adjustment of the leading calibration curve is performed using the working calibrators 146, a manufacturer's standing measurement procedure (“MSMP”) 150 and the assigned target concentration values.

Next, in step 152, the target values of product calibrators (“Product CAL”) 154 are assigned using the manufacturer's standing measurement procedure (“MSMP”) 150.

Next, in step 140, an adjustment of the leading calibration curve is performed using the product calibrators (“Product CAL”) 154, a measurement procedure on the end user's IVD medical device 110 (“Assay”) 156 and the assigned target concentration values.

Next, in step 158, the measured values for human samples are generated using the measurement procedure on the end user's IVD medical device 110 (“Assay”) 156.

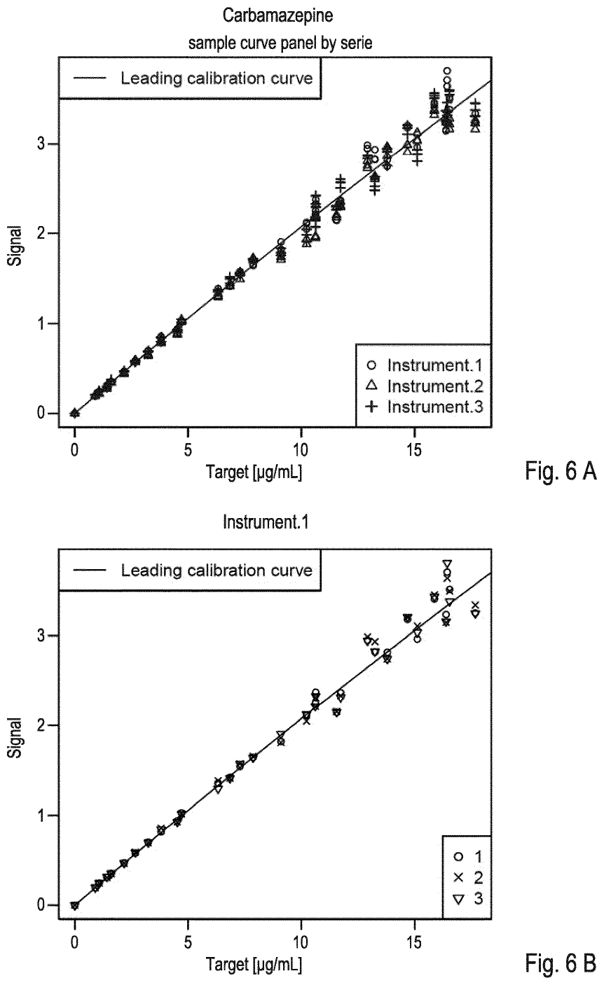

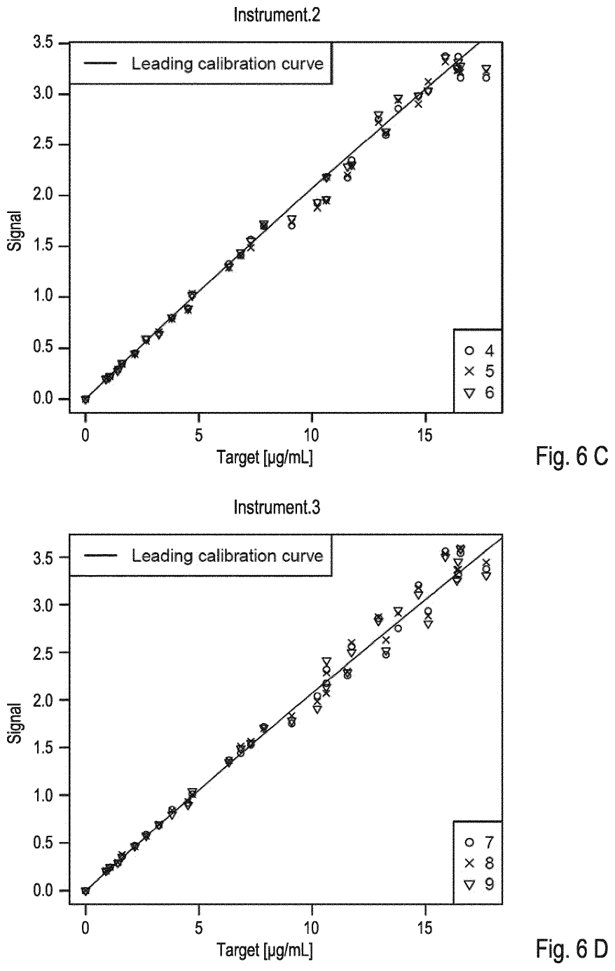

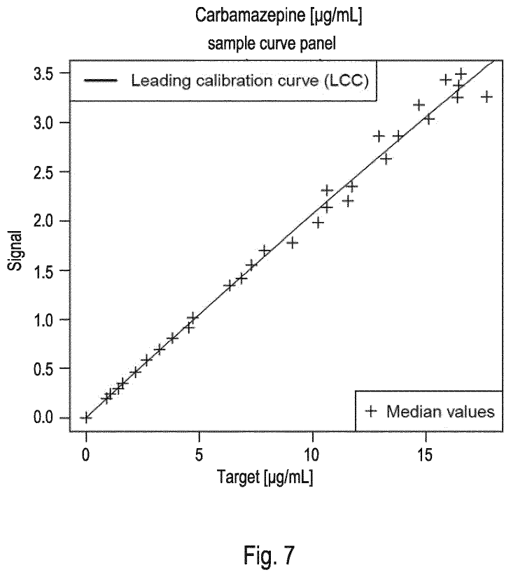

FIGS. 6A, B, C, D and FIG. 7 show experimental results of setting of the leading calibration curve 138. In particular, the signal vs the target value in [μg/ml] is shown. As material i=31 secondary calibrators 136 (in this case samples comprising Carbamazepine) were used. These i=31 secondary calibrators 136 were measured on three LC/MS instruments with three repetitions on each instrument (i.e. i=31, j=3, l=3) resulting in nine measurements (9 results (signals) per sample: sᵢⱼₗ) in total which are depicted in FIGS. 6A, B, C, and D.

FIG. 6A shows a comparison of measured signal values of the three repetitions on each secondary calibrator 136 for the three instruments, with circle denoting instrument 1, triangle denoting instrument 2 and cross denoting instrument 3. In FIGS. 6B to 6D, the experimental results of each individual instrument is shown, wherein circle, cross and triangle denote the respective repetition. In addition, the resulting leading calibration curve 138 is shown in each FIG. 6A to 6D.

The target values for the secondary calibrators 136 were pre-assigned e.g. by using a reference measurement procedure, e.g. in a reference measurement procedure for the measurand (“RMP” in FIG. 5) 134.

The following table gives an overview of the experimental results of the signal values (secondary calibrator (SPC), target value [μg/ml] (Target), minimum signal value (Min), mean signal value (Mean), median signal value (Median), maximum signal value (Max), standard deviation (SD) and coefficient of variation (CV) of signal values.

| | | | | | | | | | CV |
|---|---|---|---|---|---|---|---|---|---|
| i | j*1 | Type | Target | Min | Mean | Median | Max | SD | [%] |
| 0 | 9 | SCP | 0 | 0.000013 | 0.000102 | 0.000105 | 0.000219 | 0.000077 | 76.2 |
| 1 | 9 | SCP | 0.904 | 0.192 | 0.199 | 0.196 | 0.207 | 0.00579 | 2.9 |
| 2 | 9 | SCP | 1.08 | 0.219 | 0.236 | 0.242 | 0.248 | 0.012 | 5.1 |
| 3 | 9 | SCP | 1.42 | 0.273 | 0.294 | 0.293 | 0.313 | 0.0114 | 3.9 |
| 4 | 9 | SCP | 1.61 | 0.341 | 0.352 | 0.349 | 0.377 | 0.0105 | 3 |
| 5 | 9 | SCP | 2.18 | 0.437 | 0.459 | 0.461 | 0.473 | 0.0115 | 2.5 |
| 6 | 9 | SCP | 2.67 | 0.569 | 0.58 | 0.583 | 0.591 | 0.00761 | 1.3 |
| 7 | 9 | SCP | 3.24 | 0.637 | 0.678 | 0.691 | 0.699 | 0.0236 | 3.5 |
| 8 | 9 | SCP | 3.81 | 0.783 | 0.82 | 0.813 | 0.856 | 0.0272 | 3.3 |
| 9 | 9 | SCP | 4.52 | 0.874 | 0.91 | 0.919 | 0.939 | 0.0227 | 2.5 |
| 10 | 9 | SCP | 4.71 | 1.01 | 1.02 | 1.02 | 1.04 | 0.0115 | 1.1 |
| 11 | 9 | SCP | 6.33 | 1.29 | 1.34 | 1.35 | 1.38 | 0.0348 | 2.6 |
| 12 | 9 | SCP | 6.85 | 1.41 | 1.44 | 1.42 | 1.51 | 0.0379 | 2.6 |
| 13 | 9 | SCP | 7.29 | 1.49 | 1.55 | 1.56 | 1.57 | 0.0269 | 1.7 |
| 14 | 9 | SCP | 7.87 | 1.64 | 1.69 | 1.7 | 1.72 | 0.0333 | 2 |
| 15 | 9 | SCP | 9.1 | 1.7 | 1.79 | 1.78 | 1.9 | 0.0588 | 3.3 |
| 16 | 9 | SCP | 10.2 | 1.88 | 1.99 | 1.98 | 2.12 | 0.088 | 4.4 |
| 17 | 9 | SCP | 10.6 | 1.94 | 2.1 | 2.14 | 2.25 | 0.121 | 5.8 |
| 18 | 9 | SCP | 11.5 | 2.14 | 2.22 | 2.2 | 2.3 | 0.0685 | 3.1 |
| 19 | 9 | SCP | 10.6 | 2.18 | 2.29 | 2.31 | 2.42 | 0.087 | 3.8 |
| 20 | 9 | SCP | 11.7 | 2.29 | 2.4 | 2.35 | 2.6 | 0.121 | 5 |
| 21 | 9 | SCP | 13.2 | 2.47 | 2.67 | 2.63 | 2.93 | 0.152 | 5.7 |
| 22 | 9 | SCP | 12.9 | 2.72 | 2.86 | 2.86 | 2.98 | 0.0898 | 3.1 |
| 23 | 9 | SCP | 13.8 | 2.74 | 2.85 | 2.86 | 2.97 | 0.0923 | 3.2 |
| 24 | 9 | SCP | 15.1 | 2.8 | 2.99 | 3.03 | 3.12 | 0.105 | 3.5 |
| 25 | 9 | SCP | 14.7 | 2.91 | 3.11 | 3.18 | 3.2 | 0.116 | 3.7 |
| 26 | 9 | SCP | 16.4 | 3.14 | 3.24 | 3.25 | 3.37 | 0.0652 | 2 |
| 27 | 9 | SCP | 17.7 | 3.16 | 3.29 | 3.26 | 3.45 | 0.086 | 2.6 |
| 28 | 9 | SCP | 15.9 | 3.32 | 3.44 | 3.43 | 3.56 | 0.0804 | 2.3 |
| 29 | 9 | SCP | 16.5 | 3.16 | 3.42 | 3.49 | 3.59 | 0.163 | 4.8 |
| 30 | 9 | SCP | 16.4 | 3.23 | 3.47 | 3.37 | 3.81 | 0.199 | 5.7 |

The leading calibration curve may be determined by applying a regression fit, e.g. in this case a weighted least square fit was used. This is shown in FIG. 7. The median signal of 9 results per sample is shown on the Y-axis of FIG. 7 and the target values of the samples on the X-axis of FIG. 7. As functional form of the leading calibration curve in this example a Padé model function was used

$$y = \frac{{p}_{1} + {p}_{2} \cdot x}{1 + {p}_{3} \cdot x} ,$$

where y is signal value, x is concentration or target value and p₁, p₂ and p₃ are the parameters of the function. The following was found:

| | P1 | P2 | P3 |
|---|---|---|---|
| Pade | 0.0001048 | 0.21432 | 0.003476 |
| [μg/mL] | | | |

Subsequently to the setting of the leading calibration curve 138, the method may comprise step 144 in which the target concentration values for leading calibrators 146 are assigned.

In this experiment, eight leading calibrators (LCal) 146 were measured on three LC/MS instruments with three repetitions on each instrument (i.e. k=8, j=3, l=3), resulting in nine measurements in total and nine signals per calibrator: sₖⱼₗ^{lead,meas}. The following table gives an overview of the experimental results of the signal values (leading calibrator (LCal), minimum value (Min), mean value (Mean), median value (Median), maximum value (Max), standard deviation (SD) and coefficient of variation (CV)):

| k | j*1 | Type | Min | Mean | Median | Max | SD | CV [%] |
|---|---|---|---|---|---|---|---|---|
| 1 | 9 | LCal | 0.168 | 0.18 | 0.184 | 0.186 | 0.00771 | 4.3 |
| 2 | 9 | LCal | 0.21 | 0.218 | 0.218 | 0.226 | 0.00622 | 2.9 |
| 3 | 9 | LCal | 0.299 | 0.307 | 0.304 | 0.315 | 0.00587 | 1.9 |
| 4 | 9 | LCal | 0.772 | 0.804 | 0.805 | 0.829 | 0.0164 | 2 |
| 5 | 9 | LCal | 1.46 | 1.48 | 1.48 | 1.52 | 0.0175 | 1.2 |
| 6 | 9 | LCal | 2.06 | 2.13 | 2.14 | 2.19 | 0.0533 | 2.5 |
| 7 | 9 | LCal | 2.64 | 2.7 | 2.71 | 2.75 | 0.0421 | 1.6 |
| 8 | 9 | LCal | 3.19 | 3.34 | 3.36 | 3.51 | 0.107 | 3.2 |

The measured signals of the leading calibrators 146 were then converted into concentration values using the leading calibration curve and, if necessary, a signal adjustment function.

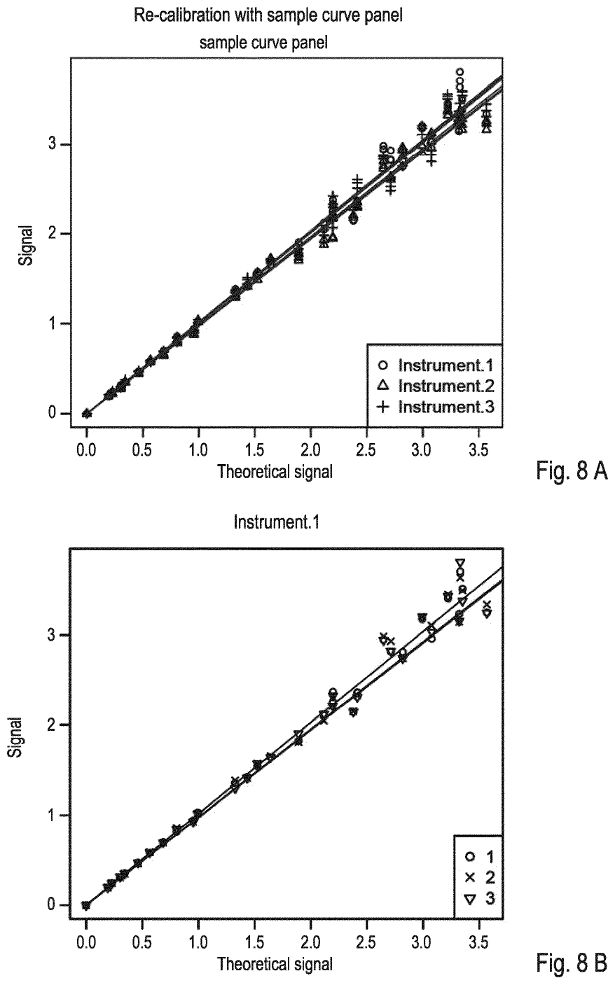

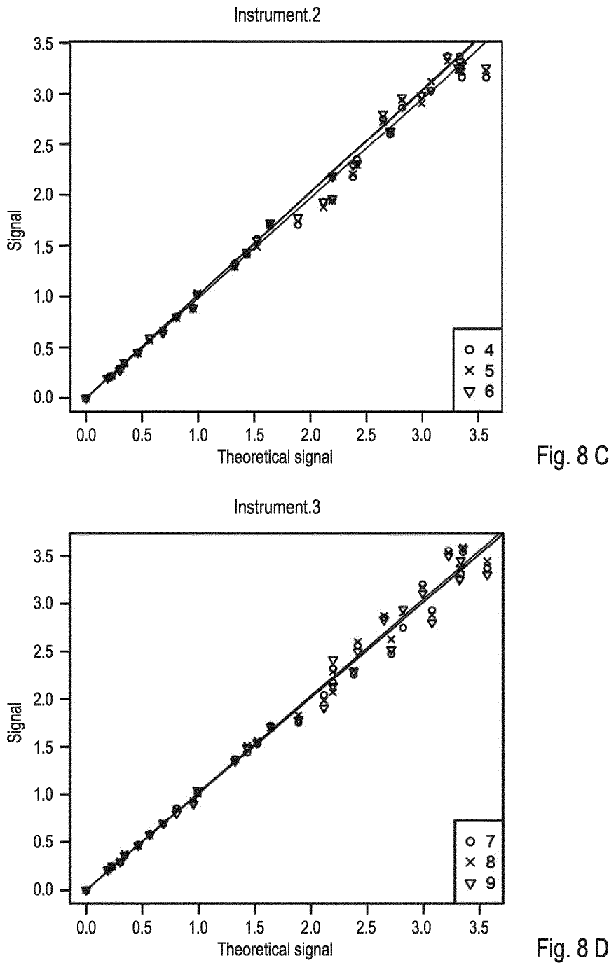

In this experiment, an adjustment of the leading calibration curve was be performed using the secondary calibrators 136. FIGS. 8A to 8D show determining of the signal adjustment function for recalibration of the leading calibration curve using sample curve panel (secondary calibrator) measurements. In particular, the samples used for determining the leading calibration curve, see description of FIG. 6, were used. The measured signals (“signal”) are shown on the Y-axis and the theoretical signals (“theoretical signal”) on the X-axis in FIGS. 8A to 8D. FIG. 8A shows a comparison of the mean values of the three repetitions for the three instruments, with circle denoting instrument 1, triangle denoting instrument 2 and cross denoting instrument 3. In FIGS. 8B to 8D, the experimental results of each individual instrument is shown, wherein circle, cross and triangle denote the respective repetition. The fit result is shown in each Figure. In FIGS. 8B to 8D, the following intercepts and slopes were determined:

| Sample | | |
|---|---|---|
| ID | Intercept | Slope |
| 1 | −0.0000323 | 0.98713 |
| 2 | −0.0001081 | 1.02579 |
| 3 | −0.0001104 | 1.03035 |
| 4 | −0.0000013 | 1.01675 |
| 5 | 0.0000112 | 0.98578 |
| 6 | 0.0000554 | 0.98932 |
| 7 | 0.0000732 | 0.99335 |
| 8 | 0.0000778 | 0.98383 |
| 9 | 0.0000918 | 0.99455 |

This result was used for determining the respective concentration values for the leading calibrators and the median of these concentrations is used as target value for the leading calibrators 146. In particular, as target concentration values of the leading calibrators 146 the median concentration in [μg/ml] of the 9 read concentrations (from 9 signals per calibrator) were used. The following table gives an overview of the experimental concentration results in [μg/mL]:

| | | | | Median/ | | | CV |
|---|---|---|---|---|---|---|---|
| SampleID | N | Min | Mean | Target | Max | SD | [%] |
| 1 | 9 | 0.797 | 0.84 | 0.848 | 0.867 | 0.0246 | 2.9 |
| 2 | 9 | 0.997 | 1.02 | 1.02 | 1.05 | 0.0183 | 1.8 |
| 3 | 9 | 1.38 | 1.44 | 1.45 | 1.49 | 0.0361 | 2.5 |
| 4 | 9 | 3.71 | 3.8 | 3.81 | 3.87 | 0.0597 | 1.6 |
| 5 | 9 | 6.85 | 7.09 | 7.04 | 7.39 | 0.167 | 2.4 |
| 6 | 9 | 10.1 | 10.3 | 10.3 | 10.5 | 0.115 | 1.1 |
| 7 | 9 | 12.7 | 13.2 | 13.2 | 13.4 | 0.228 | 1.7 |
| 8 | 9 | 16.1 | 16.5 | 16.5 | 17.1 | 0.302 | 1.8 |

Next, the method may comprise step 152, in which the target concentration values for product calibrators 154 are assigned.

In this experiment, two product calibrators 154 were measured on three LC/MS instruments with three repetitions on each instrument (i.e. k=2, j=3, l=3), resulting in nine measurements in total and nine signals per calibrator. The following table gives an overview of the experimental results of the signal values (product calibrator (PCal), minimum value (Min), mean value (Mean), median value (Median), maximum value (Max), standard deviation (SD) and coefficient of variation (CV)):

| | | | | | | | CV |
|---|---|---|---|---|---|---|---|
| SampleID | N | Min | Mean | Median | Max | SD | [%] |
| PCal1 | 9 | 0.321 | 0.343 | 0.344 | 0.367 | 0.0152 | 4.4 |
| PCal2 | 9 | 2.47 | 2.61 | 2.61 | 2.74 | 0.0913 | 3.5 |

The measured signals of the product calibrators 154 were then converted into concentration values using the leading calibration curve and a signal adjustment function.

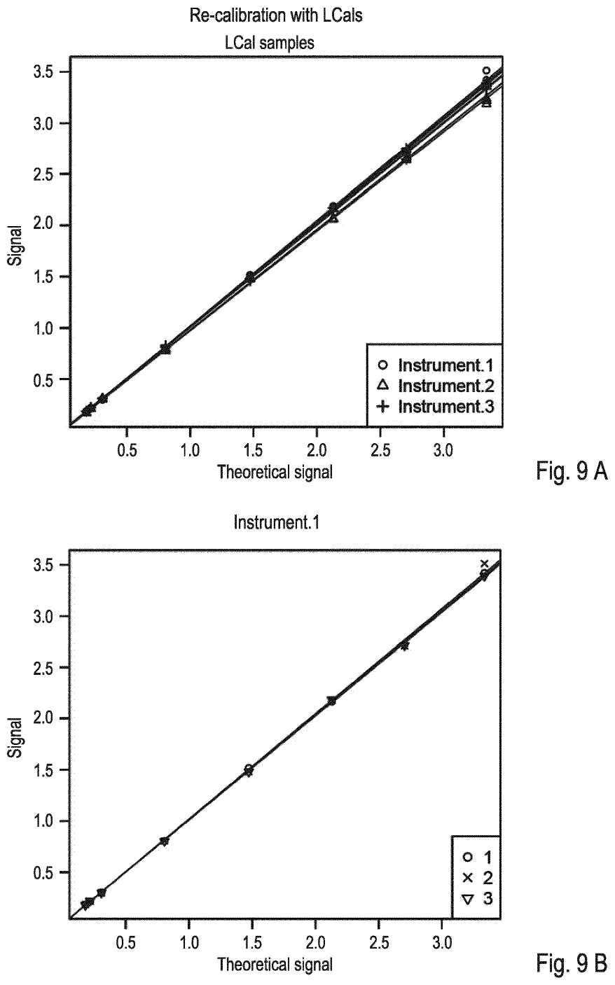

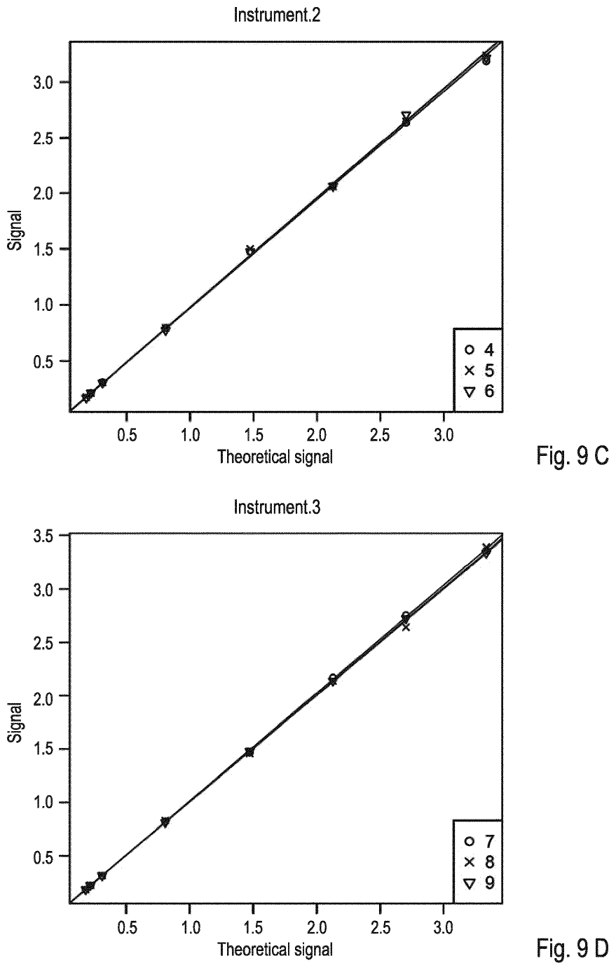

FIGS. 9A to 9D show determining of the signal adjustment function for recalibration of the leading calibration curve using the leading calibrators 146 and the determined target values of the leading calibrators 146. The measured signals (“signal”) are shown on the Y-axis and the theoretical signals (“theoretical signal”) on the X-axis in FIGS. 9A to 9D. FIG. 9A shows a comparison of the mean values of the three repetitions on each leading calibrators 146 for the three instruments, with circle denoting instrument 1, triangle denoting instrument 2 and cross denoting instrument 3. In FIGS. 9B to 9D, the experimental results of each individual instrument is shown, wherein circle, cross and triangle denote the respective repetition. The fit result is shown in each Figure. In FIGS. 9B to 9D, the following intercepts and slopes were determined:

| Lcal. | | |
|---|---|---|
| Serie | Intercept | Slope |
| 1 | 0.004825 | 0.98049 |
| 2 | 0.008013 | 0.97348 |
| 3 | 0.004905 | 0.98536 |
| 4 | −0.00064 | 1.02901 |
| 5 | 0.000471 | 1.02044 |
| 6 | 0.005589 | 1.01754 |
| 7 | −0.00075 | 0.98856 |
| 8 | −0.00663 | 1.0021 |
| 9 | −0.00602 | 0.99879 |

This result was used for determining the respective concentration values and the median of these concentrations is used as target value for the product calibrators 154. In particular, as target concentration values of the product calibrators 154 the median concentration in [μg/ml] of the 9 read concentrations (from 9 signals per calibrator) were used. The following table gives an overview of the experimental concentration results in [μg/mL]:

| | | | | | Median/ | | | CV |
|---|---|---|---|---|---|---|---|---|
| SampleID | N | Type | Min | Mean | Target | Max | SD | [%] |
| PCal1 | 9 | PCal | 1.56 | 1.61 | 1.59 | 1.71 | 0.0572 | 3.6 |
| PCal2 | 9 | PCal | 12.2 | 12.7 | 12.7 | 13.1 | 0.287 | 2.3 |

Next, the method may comprise step 158, in which the final measured concentration values for human samples and controls (in particular QC samples and/or marker samples “m.6.human sample with result”) are assigned.

In this experiment, three human samples were measured on three LC/MS instruments with three repetitions on each instrument (i.e. k=3, j=3, l=3), resulting in nine measurements in total and nine signals per sample. The following table gives an overview of the experimental results of the signal values (control samples (QC), minimum value (Min), mean value (Mean), median value (Median), maximum value (Max), standard deviation (SD) and coefficient of variation (CV)) and its pre assigned target values in [μg/mL]:

| | | | | | | | | | CV |
|---|---|---|---|---|---|---|---|---|---|
| SampleID | N | Type | Target | Min | Mean | Median | Max | SD | [%] |
| QC1 | 9 | QC | 2.03 | 0.404 | 0.425 | 0.425 | 0.441 | 0.0123 | 2.9 |
| QC2 | 9 | QC | 7.51 | 1.57 | 1.61 | 1.6 | 1.68 | 0.0356 | 2.22 |
| QC3 | 9 | QC | 14.1 | 2.75 | 2.85 | 2.82 | 3.06 | 0.0994 | 3.5 |

The measured signals of the human samples were then converted into concentration values using the leading calibration curve and a signal adjustment function.

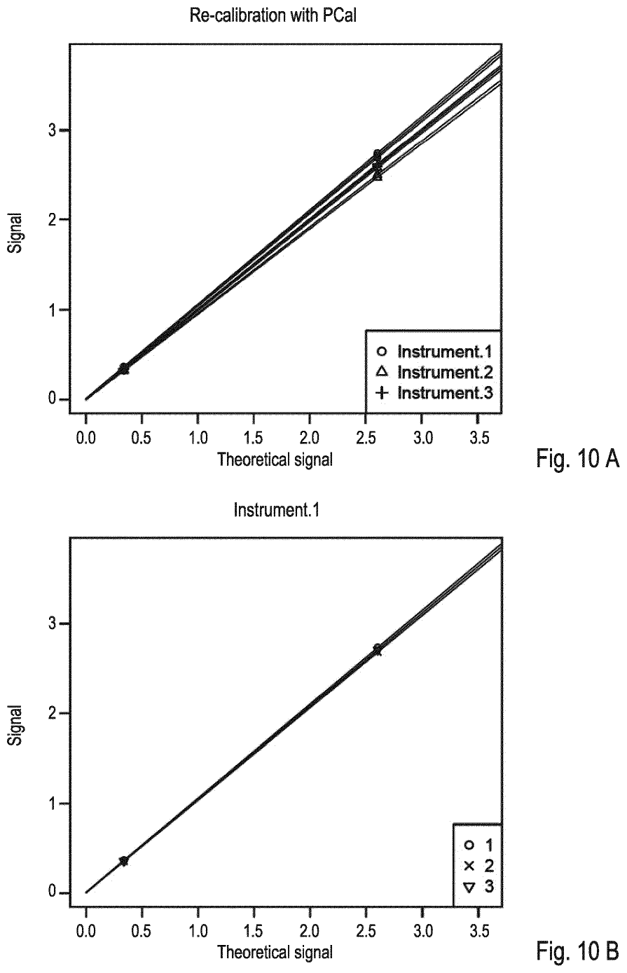

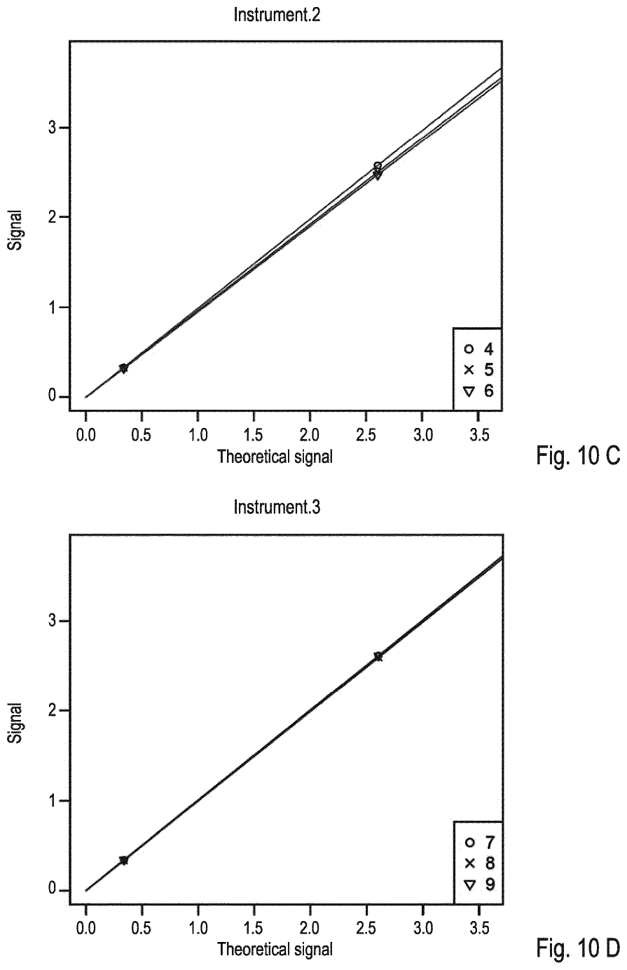

FIGS. 10A to 10D show determining of the signal adjustment function for recalibration of the leading calibration curve using the product calibrators calibrators 146 and the determined target values of the leading calibrators 154. The measured signals (“signal”) are shown on the Y-axis and the theoretical signals (“theoretical signal”) on the X-axis in FIGS. 10A to 10D. FIG. 10A shows a comparison of the mean values of the three repetitions on each product calibrators 154 for the three instruments, with circle denoting instrument 1, triangle denoting instrument 2 and cross denoting instrument 3. In FIGS. 10B to 10D, the experimental results of each individual instrument is shown, wherein circle, cross and triangle denote the respective repetition. The fit result is shown in each Figure. In FIGS. 10B to 10D, the following intercepts and slopes were determined:

| System | Stream | Intercept | Slope |
|---|---|---|---|
| Instrument, | 1 | −0.01152 | 0.95558 |
| 1 | | | |
| Instrument, | 2 | −0.00697 | 0.97205 |
| 1 | | | |
| Instrument, | 3 | −0.00473 | 0.96256 |
| 1 | | | |
| Instrument, | 1 | 0.003305 | 1.01117 |
| 2 | | | |
| Instrument, | 2 | −0.002 | 1.04354 |
| 2 | | | |
| Instrument, | 3 | 0.001018 | 1.05473 |
| 2 | | | |
| Instrument, | 1 | −0.0039 | 0.99773 |
| 3 | | | |
| Instrument, | 2 | 0.000107 | 1.00411 |
| 3 | | | |
| Instrument, | 3 | −0.00401 | 0.99803 |
| 3 | | | |

This result was used for determining the respective concentration values and the mean of these concentrations is used to calculate the relative recovery to its pre assigned target value. The following table gives an overview of the experimental results in [μg/mL]:

| | | | | | | | | | | | Re- |
|---|---|---|---|---|---|---|---|---|---|---|---|
| | | | | | | | | | CV | Signal. | cov. |
| SampleID | N | Type | Target | Min | Mean | Median | Max | SD | [%] | CV | [%] |
| QC1 | 9 | QC | 2.03 | 1.91 | 1.98 | 1.98 | 2.04 | 0.0374 | 1.89 | 2.9 | 97.6 |
| QC2 | 9 | QC | 7.51 | 7.13 | 7.68 | 7.62 | 8.18 | 0.344 | 4.48 | 2.22 | 102.3 |
| QC3 | 9 | QC | 14.1 | 12.8 | 13.9 | 14.1 | 14.9 | 0.764 | 5.49 | 3.49 | 98.7 |

### LIST OF REFERENCE NUMBERS

110 in vitro diagnostic medical device
111 mass spectrometry device
112 processing device
113 quadrupol mass analyzer
114 detector
115 ionization source
116 hardware part
118 processor
120 data storage device
122 “p.1. Fit for purpose measurement procedures for purity assessment (e.g. qNMR, mass balance)”
124 target value assignment
126 “m.1. certified primary reference material (CRM)”
128 calibration step
130 “p.2. primary reference measurement procedure for calibrator preparation (e.g. gravimetric preparation)”
132 “m.2. primary calibrator prepared as solution of m.1 in suitable solvent”
134 “p.3. primary reference measurement procedure for the measurand”
136 “m.3. secondary calibrator (reference samples or pools or human samples)”
138 Setting of the leading calibration curve
140 Adjustment of the leading calibration curve
142 “p.4. manufacturer's selected measurement procedure”
144 Target value assignment of leading calibrators
146 “m.4. manufacturer's working calibrator (leading calibrator)”
148 Adjustment of the leading calibration curve by leading calibrators
150 “p.5. manufacturer's standing measurement procedure”
152 “target value assignment of product calibrator”
154 “m.5. end user IVD MD calibrator (product calibrator)”
156 “p.6. end-users IVD MD”
157 leading calibration curve
157a leading calibration curve for analyte A
157b leading calibration curve for analyte B
157c leading calibration curve for analyte C
158 target value assignment of human samples
159 curve illustrating effect of signal adjustment function
160 “m.6. human samples with results”
161 concentration in arbitrary units
162 methods for establishing metrological traceability for at least one in vitro diagnostic medical device
163 signal in arbitrary units
164 available: leading calibration curve (LCC), pre-assigned target concentration value of first calibrator sample and measured signal value of the first calibrator sample
166 first calibrator samples
168 second calibrator samples
170 application of leading calibration curve or its inverse
172 determination of adjustment function
174 assignment of target concentration value to second calibrator sample
176 consecutive application of the inverse of the adjustment function (or the adjustment function) and the inverse of the leading calibration curve or vice versa

1. A method for establishing metrological traceability for at least one in vitro diagnostic medical device, wherein the method comprises a sequence of calibration and adjustment steps, wherein an outcome of each step depends on the outcome of the previous step,
wherein the method comprises providing a leading calibration curve, wherein the leading calibration curve fₚ describes a relationship of at least one concentration c of at least one analyte in at least one sample with a signal s of the sample measured with the in vitro diagnostic medical device, wherein the leading calibration curve fₚ is a parametrized function fₚ(c, \hat{p}₁, . . . , \hat{p}_{P}) with parameters \hat{p}₁, . . . , \hat{p}_{P} being a set of parameters of the leading calibration curve and P≥1,
wherein in each adjustment step a signal adjustment function gᵣ with r being a set of parameters of the signal adjustment function, describing a relationship between measured and theoretical signal values, is determined by determining the relationship between measured signal values of first calibrator samples and theoretical signal values of the first calibrator samples derived from the leading calibration curve, wherein the theoretical signal values are determined by applying the leading calibration curve using pre-assigned target concentration values cᵢ of the first calibrator samples,
wherein each adjustment step comprises assigning at least one target concentration value from measured signal values of at least one second calibrator sample, wherein the assigning the at least one target concentration value comprises determining at least one theoretical signal value of the second calibrator sample by applying the signal adjustment function determined in the previous adjustment step or the inverse of the signal adjustment function determined in the previous adjustment step to the measured signal values of the second calibrator sample and applying the inverse leading calibration curve f_{P}⁻¹ to the theoretical signal value of the second calibrator sample.

2. The method according to claim 1, wherein the sequence of calibration and adjustment steps comprises a first calibration and adjustment step using a fit for purpose measurement procedure for purity assessment, wherein the sequence of calibration and adjustment steps further comprises a second calibration and adjustment step using a primary reference measurement procedure for calibrator preparation on at least one certified primary reference material, wherein the sequence of calibration and adjustment steps further comprises a third calibration and adjustment step using a primary reference measurement procedure for a measurand on at least one primary calibrator, and a forth calibration and adjustment step using a manufacturer selected measurement procedure on at least one secondary calibrator.

3. The method according to claim 2, wherein the leading calibration curve is determined by using at least one primary calibrator, wherein at least one target concentration value of the primary calibrator is established based on the primary reference measurement procedure for a calibrator preparation.

4. The method according to claim 1, wherein the leading calibration curve is determined by using at least one secondary calibrator, wherein at least one target concentration value of the secondary calibrator is established based on the primary reference measurement procedure for a measurand.

5. The method according to claim 1, wherein the assigned target concentration values of the second calibrator samples are usable in a subsequent adjustment step for determining the signal adjustment function.

6. The method according to claim 1, wherein the leading calibration curve is unchanged over two or more adjustment steps.

7. The method according to claim 1, wherein the signal adjustment function is a function g(sᵢ^{theo}, r₁, . . . , r_{R}) with r₁, . . . , r_{R} being a set of parameters of the signal adjustment function, R≥1 and i being the first calibrator samples i=1, . . . I, I≥1, wherein the signal adjustment function connects theoretical signals of the leading calibration curve sᵢ^{theo}=f(cᵢ, \hat{p}₁, . . . , \hat{p}_{P}), with i≥1 and cᵢ being the pre-assigned target concentration values of the first calibrator samples, with measured signal values sᵢⱼₗ^{meas} of the first calibrator samples, wherein j denotes at least one of an instrument or a hardware part of the in vitro diagnostic medical device j=1, . . . , J and J≥1, and l denoting the repeat l=1, . . . , L, and L≥1, wherein determining the signal adjustment function comprises measuring the signal values sᵢⱼₗ^{meas} of the first calibrator samples, calculating the theoretical signals sᵢ^{theo}, and fitting the signal adjustment function thereby determining the fitted parameters \hat{r}₁, . . . , \hat{r}_{R}.

8. The method according to claim 1, wherein the assignment of the target concentration value comprises measuring signal values sₖⱼₗ^{meas} of the second calibrator sample k, with k=1, . . . , K and K≥1 using the in vitro diagnostic medical device and transforming the measured signal values sₖⱼₗ^{meas} into target concentration values cₖⱼₗ by applying the following inverse functions consecutively:
$${s}_{kjl}^{theo} = {g}_{1}^{- 1} ( {s}_{kjl}^{meas} , {( {\hat{r}}_{1} , … , {\hat{r}}_{R} )}_{j} ) ; and$$
$${c}_{kjl} = {f}^{- 1} ( {s}_{kjl}^{theo} , {\hat{p}}_{1} , … , {\hat{p}}_{P} ) .$$

9. The method according to claim 1, wherein the signal adjustment function is a function g(sᵢ^{theo}, r₁, . . . , r_{R}) with r₁, . . . , r_{R} being a set of parameters of the signal adjustment function, R≥1 and i being the first calibrator samples i=1, . . . I, I≥1, wherein the signal adjustment function connects measured signal values of the first calibrator samples with the theoretical signals of the first calibrator samples derived from the preassigned target concentration values and the leading calibration function, wherein the assignment of the target concentration values of the second calibrator sample is determined by applying the signal adjustment function.

10. The method according to claim 1, wherein the in vitro diagnostic medical device is a mass spectrometry device.

11. A method for establishing metrological traceability for at least one in vitro diagnostic medical device, wherein the method comprises a sequence of calibration steps and adjustment steps, wherein an outcome of each step depends on the outcome of the previous step,
wherein the method comprises providing a leading calibration curve, wherein the leading calibration curve fₚ describes a relationship of at least one concentration c of at least one analyte in at least one sample with a signal s of the sample measured with the in vitro diagnostic medical device, wherein the leading calibration curve fₚ is a parametrized function fₚ(c, \hat{p}₁, . . . , \hat{p}_{P}) with parameters \hat{p}₁, . . . , \hat{p}_{P} being a set of parameters of the leading calibration curve and P≥1,
wherein in each adjustment step a concentration adjustment function hₛ with s being a set of parameters of the concentration adjustment function, describing a relationship between a theoretical concentration value of measured signal values of first calibrator samples and a preassigned target concentration value of the first calibrator samples, is determined, wherein the theoretical concentration value of the first calibrator samples is determined by applying the inverse of the leading calibration curve f_{P}⁻¹ using the measured signal values of the first calibrator samples,
wherein the assignment of the target concentration value comprises applying the inverse of the leading calibration curve f_{P}⁻¹ using measured signal values of second calibrator samples, thereby obtaining theoretical concentration values, and applying one of the concentration adjustment function hₛ or the inverse of the concentration adjustment function hₛ⁻¹ on said theoretical concentration values, thereby obtaining the target concentration values of the second calibrator samples.

12. The method according to claim 11, wherein the concentration adjustment function connects pre-assigned target concentration values of the first calibrator samples with the theoretical concentration values of the measured signal of the first calibrator samples, and the assignment of the target concentration values of second calibrator samples is determined by applying the concentration adjustment function.

13. A processing device, wherein the processing device is configured for at least one of retrieving or storing at least one pre-determined leading calibration curve fₚ, wherein the processing device is further configured for storing a set of parameters \hat{p}₁, . . . , \hat{p}_{P} of the leading calibration curve fₚ, wherein P is a positive integer, wherein the leading calibration curve fₚ describes a relationship of at least one concentration c of at least one analyte in at least one sample with a signal s of the sample measured with an in vitro diagnostic medical device, wherein the leading calibration curve fₚ is a parametrized function fₚ(c, \hat{p}₁, . . . , \hat{p}_{P}), and wherein the processing device performs the method for establishing metrological traceability for at least one in vitro diagnostic medical device according to claim 1.

14. A kit comprising an in vitro diagnostic medical device, a set of product calibrators and their target concentration values, wherein the target concentration values of said product calibrators are assigned by using the method according to claim 1.

15. (canceled)

16. (canceled)

17. A processing device, wherein the processing device is configured for at least one of retrieving or storing at least one pre-determined leading calibration curve fₚ, wherein the processing device is further configured for storing a set of parameters \hat{p}₁, . . . , \hat{p}_{P} of the leading calibration curve fₚ, wherein P is a positive integer, wherein the leading calibration curve fₚ describes a relationship of at least one concentration c of at least one analyte in at least one sample with a signal s of the sample measured with an in vitro diagnostic medical device, wherein the leading calibration curve fₚ is a parametrized function fₚ(c, \hat{p}₁, . . . , \hat{p}_{P}), and wherein the processing device performs the method for establishing metrological traceability for at least one in vitro diagnostic medical device according to claim 11.

18. A kit comprising an in vitro diagnostic medical device, a set of product calibrators and their target concentration values, wherein the target concentration values of said product calibrators are assigned by using the method according to claim 11.
